(19) United States
(12) Patent Application Publication
(10) Pub. No.: US 2024/0182551 A1
(43) Pub. Date: Jun. 6, 2024

(54) CROSS-REACTIVE MONOCLONAL ANTIBODIES AGAINST CORONAVIRUSES

(71) Applicant: THE SCRIPPS RESEARCH INSTITUTE, La Jolla, CA (US)

(72) Inventors: Raiees Andrabi, San Diego, CA (US); Ge Song, San Diego, CA (US); Wanting He, San Diego, CA (US); Thomas Rogers, Del Mar, CA (US); Dennis R. Burton, La Jolla, CA (US)

(21) Appl. No.: 18/553,216

(22) PCT Filed: Mar. 31, 2022
(86) PCT No.: PCT/US22/22759

§ 371 Date: Sep. 29, 2023

Related U.S. Application Data
(60) Provisional application No. 63/170,105, filed on Apr. 2, 2021.

(51) Int. Cl. C07K 16/10; A61K 39/00

(52) U.S. Cl. CPC C07K 16/1003; A61K 2039/505; C07K 2317/33; C07K 2317/55; C07K 2317/565

(57) ABSTRACT

The invention provides antibodies and related antibody agents that are cross-reactive with the spike proteins of multiple human coronaviruses. Also provided in the invention are methods and kits of using such antibodies in various diagnostic and therapeutic applications. In one aspect, the invention provides novel antibodies or antigen-binding fragments thereof that specifically bind to a human coronavirus spike protein.

### CROSS-REFERENCE TO RELATED APPLICATIONS

The subject patent application claims the benefit of priority to U.S. Provisional Patent Application No. 63/170,105 (filed Apr. 2, 2021; now pending). The full disclosure of the priority application is incorporated herein by reference in its entirety and for all purposes.

### STATEMENT OF GOVERNMENT SUPPORT

This invention was made with government support under grant numbers AI144462, AI132317 and AI073148 awarded by the National Institutes of Health. The government has certain rights in the invention.

### BACKGROUND OF THE INVENTION

There are increasing virus mutants of SARS-CoV-2 emerging continuously and also a potential threat of SARS-like vial spillovers through zoonotic transmissions from animal reservoirs. It is therefore important to develop therapeutic and preventive regimens that could be employed to effectively counter infections by different coronaviruses or different viral variants. Identification of cross-reactive mAbs against human coronaviruses (HCoVs) could greatly facilitate development of such regimens. The instant invention is directed to addressing this and other unmet needs in the art.

### SUMMARY OF THE INVENTION

In one aspect, the invention provides novel antibodies or antigen-binding fragments thereof that specifically bind to a human coronavirus (HCoV) spike (S) protein. These antibodies bind to the S protein with the same binding specificity as that of an antibody containing heavy chain CDR1, CDR2 and CDR3 sequences and light chain CDR1, CDR2 and CDR3 sequences respectively set forth as (1) SEQ ID NOs:10-13, EDK, and SEQ ID NO:14 (CC40.8), (2) SEQ ID NOs:15-18, KVS, and SEQ ID NO:19 (CC40.5), (3) SEQ ID NOs:20-23, KVS, and SEQ ID NO:24 (CC9.1/9.2), or (4) SEQ ID NOs:25-28, WAS, and SEQ ID NO:29 (CC9.3). In some embodiments, the antibody or antigen-binding fragment of the invention has a heavy chain variable region containing CDR1-3 sequences that are respectively identical to SEQ ID NOs:10-13; SEQ ID NOs:15-17; SEQ ID NOs:20-22; or SEQ ID NOs:25-27. In some of these embodiments, the antibody or antigen-binding fragment of the invention also has a light chain variable region containing CDR1-3 sequences that are respectively identical to SEQ ID NO:13, EDK, and SEQ ID NO:14; SEQ ID NO:18, KVS, and SEQ ID NO:19; SEQ ID NO:23, KVS, and SEQ ID NO:24; or (4) SEQ ID NO:28, WAS, and SEQ ID NO:29. In some embodiments, the antibody or antigen-binding fragment of the invention has a light chain variable region containing CDR1-3 sequences that are respectively identical to SEQ ID NO:13, EDK, and SEQ ID NO:14; SEQ ID NO:18, KVS, and SEQ ID NO:19; SEQ ID NO:23, KVS, and SEQ ID NO:24; or (4) SEQ ID NO:28, WAS, and SEQ ID NO:29.

In some embodiments, the antibody or antigen-binding fragment of the invention has heavy chain CDR1, CDR2 and CDR3 sequences and light chain CDR1, CDR2 and CDR3 sequences that are respectively identical to (1) SEQ ID NOs:10-13, EDK, and SEQ ID NO:14 (CC40.8), or conservatively modified variant thereof, (2) SEQ ID NOs:15-18, KVS, and SEQ ID NO:19 (CC40.5), or conservatively modified variant thereof, (3) SEQ ID NOs:20-23, KVS, and SEQ ID NO:24 (CC9.1/9.2), or conservatively modified variant thereof, or (4) SEQ ID NOs:25-28, WAS, and SEQ ID NO:29 (CC9.3), or conservatively modified variant thereof. In some embodiments, the antibody or antigen-binding fragment of the invention has heavy chain CDR1, CDR2 and CDR3 sequences and light chain CDR1, CDR2 and CDR3 sequences that are respectively identical to (1) SEQ ID NOs:10-13, EDK, and SEQ ID NO: 14 (CC40.8); (2) SEQ ID NOs:15-18, KVS, and SEQ ID NO:19 (CC40.5); (3) SEQ ID NOs:20-23, KVS, and SEQ ID NO:24 (CC9.1/9.2); or (4) SEQ ID NOs:25-28, WAS, and SEQ ID NO:29 (CC9.3). In some of these embodiments, the antibody or antigen-binding fragment has a heavy chain variable region and a light chain variable region that are substantially identical to or conservatively modified variants of (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively. In some of these embodiments, the antibody or antigen-binding fragment has a heavy chain variable region and a light chain variable region that are at least 95% identical to (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively. In some embodiments, the antibody or antigen-binding fragment has a heavy chain variable region and a light chain variable region that are at least 99% identical to (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively. In some embodiments, the antibody or antigen-binding fragment has a heavy chain variable region and a light chain variable region that are respectively identical to (1) SEQ ID NOs:1 and 2; (2) SEQ ID NOs:3 and 4; (3) SEQ ID NOs:5 and 6; (4) SEQ ID NOs:5 and 7; or (5) SEQ ID NOs:8 and 9.

In a related aspect, the invention provides polynucleotide sequences that encode (1) an antibody heavy chain variable region containing CDR1-3 sequences (HCDR1-3) that are respectively identical to SEQ ID NOs:10-13; SEQ ID NOs:15-17; SEQ ID NOs:20-22; or SEQ ID NOs:25-27; and/or (2) an antibody light chain variable region containing CDR1-3 sequences (LCDR1-3) that are respectively identical to SEQ ID NO:14, EDK, and SEQ ID NO:14; SEQ ID NO:18, KVS, and SEQ ID NO: 19; SEQ ID NO:23, KVS, and SEQ ID NO:24; or (4) SEQ ID NO:28, WAS, and SEQ ID NO:29. In some of these embodiments, the polynucleotide sequence encodes an antibody heavy chain variable region and an antibody light chain variable region, wherein the HCDR1-3 and the LCDR1-3 are respectively identical to (1) SEQ ID NOs:10-13, EDK, and SEQ ID NO:14, (2) SEQ ID NOs:15-18, KVS, and SEQ ID NO:19, (3) SEQ ID NOs:20-23, KVS, and SEQ ID NO:24, or (4) SEQ ID NOs:25-28, WAS, and SEQ ID NO:29. In some of these embodiments, the encoded HCDR1-3 and LCDR1-3 are respectively identical to SEQ ID NOs:10-13, EDK, and SEQ ID NO: 14. In some of these embodiments, the encoded heavy chain variable region and light chain variable region are substantially identical to or conservatively modified variants of (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively. In some of these embodiments, the encoded heavy chain variable region and light chain variable region are respectively identical to (1) SEQ ID NOs:1 and 2; (2) SEQ ID NOs:3 and 4; (3) SEQ ID NOs:5 and 6; (4) SEQ ID NOs:5 and 7; or (5) SEQ ID NOs:8 and 9.

In a related aspect, the invention provides pharmaceutical compositions that contain a therapeutically effective amount of the novel antibody or antigen-binding fragment disclosed herein and a pharmaceutically acceptable carrier. In some of these embodiments, the pharmaceutical composition contains an antibody or antigen-binding fragment that has HCDR1-3 and LCDR1-3 respectively identical to SEQ ID NOs:10-13, EDK, and SEQ ID NO:14. In some of these embodiments, the antibody or antigen-binding fragment in the pharmaceutical composition has a heavy chain variable region and a light chain variable region respectively identical to SEQ ID NOs:1 and 2. In another related aspect, the invention provides kits that contain a novel antibody or antigen-binding fragment disclosed herein.

In another aspect, the invention provides therapeutic methods for treating or ameliorating symptoms associated with coronavirus infections in a subject. These methods entail administering a pharmaceutical composition disclosed herein to a subject afflicted with infection by one or more human coronaviruses (HCoVs). In some of these methods, the subject is afflicted with infection of SARS-CoV-2. In some embodiments, the administered pharmaceutical composition contains an antibody or antigen-binding fragment that has heavy chain CDR1, CDR2 and CDR3 sequences and light chain CDR1, CDR2 and CDR3 sequences respectively identical to SEQ ID NOs:10-13, EDK, and SEQ ID NO:14.

In still another aspect, the invention provides methods for diagnosing a coronavirus infection in a human subject. These methods involve (a) obtaining a biological sample from the subject, and (b) contacting the sample with a novel antibody or antigen-binding fragment disclosed herein to detect a specific binding between an antigen in the sample and the antibody or antigen-binding fragment. In some embodiments, the biological sample to be obtained from the subject is a blood sample or a saliva sample. In some embodiments, the employed antibody or antigen-binding fragment has heavy chain HCDRs 1-3 and LCDRs 1-3 sequences that are respectively identical to SEQ ID NOs:10-13, EDK, and SEQ ID NO:14.

A further understanding of the nature and advantages of the present invention may be realized by reference to the remaining portions of the specification and claims.

### DESCRIPTION OF THE DRAWINGS

FIG. 1. Reactivity of COVID and pre-pandemic human sera with cell surface-expressed human coronaviruses spikes and their soluble S-protein versions. Panel a. Heatmap showing cell-based flow cytometry binding (CELISA) of COVID and pre-pandemic donor sera with 293T cell surface-expressed full-length spike proteins from β-(SARS-CoV-2, SARS-CoV-1 (aka SARS-CoV), MERS-CoV, HCoV-HKU1, HCoV-OC43) and α-(HCoV-NL63 and HCoV-229E) human coronaviruses (HCoVs). Sera were titrated (6 dilutions—starting at 1:30 dilution) and the extent of binding to cell surface-expressed HCoVs was recorded by % positive cells, as detected by PE-conjugated anti-human-Fc secondary Ab using flow cytometry. Area-under-the-curve (AUC) was calculated for each binding titration curve and the antibody titer levels are as indicated. Binding of sera to vector-only plasmid (non-spike) transfected 293T cells served as a control for non-specific binding. Panel b. ELISA binding of COVID and pre-pandemic donor sera to soluble S-proteins from b-(SARS-CoV-2, SARS-CoV-1, MERS-CoV, HCoV-HKU1, HCoV-OC43) and a-(HCoV-NL63 and HCoV-229E) HCoVs. Serum dilutions (8 dilutions—starting at 1:30 dilution) were titrated against the S-proteins and the binding was detected as OD405 absorbance. AUC representing the extent of binding was calculated from binding curves of COVID (left) and pre-pandemic (right) sera with S-proteins and comparisons of antibody binding titers are shown. Binding of sera with each protein is shown as scatter dot plots with a line at median. Binding to BSA served as a control for non-specific binding by the sera. The serum binding experiments were carried out in duplicate and repeated independently at least once for reproducibility. Statistical comparisons between two groups were performed using a Mann-Whitney two-tailed test, (**p<0.01; ***p<0.001, ****p<0.0001; ns-p>0.05).

FIG. 2. BioLayer Interferometry binding of COVID and pre-pandemic serum antibodies to SARS-CoV-2 and endemic HCoV S-proteins. Panel a. Heatmap summarizing the apparent BLI binding off-rates (k_{off} (1/s)) of the COVID and pre-pandemic human serum antibodies to SARS-CoV-2 S and endemic b-HCoV, HCoV-HKU1 and α-HCoV, HCoV-NL63 S-proteins. Biotinylated HCoV S-proteins (100 nM) were captured on streptavidin biosensors to achieve binding of at least 1 response unit. The S-protein-immobilized biosensors were immersed in 1:40 serum dilution solution with serum antibodies as the analyte and the association (120 s; 180-300) and dissociation (240 s; 300-540) steps were conducted to detect the kinetics of antibody-protein interaction. k_{off} (1/s) dissociation rates for each antibody-antigen interaction are shown. Panel b. Off-rates for binding of serum antibodies from COVID donors and from pre-pandemic donors to SARS-CoV-2 S and endemic HCoV, HCoV-HKU1 and HCoV-NL63, S proteins. Significantly lower dissociation off-rates are observed for COVID compared to pre-pandemic sera. Statistical comparisons between the two groups were performed using a Mann-Whitney two-tailed test, (****p<0.0001).

FIG. 3. SARS-CoV-2 S and endemic HCoV S-protein specific cross-reactive IgG+ memory B cells from COVID donors and isolation and characterization of mAbs. Panels a-b. Flow cytometry analysis showing the single B cell sorting strategy for COVID representative donor CC9 and frequencies of SARS-CoV-2 S and endemic b-HCoV, HCoV-HKU1 and α-HCoV, HCoV-NL63 S-protein specific memory B cells in 8 select COVID donors. The B cells were gated as SSL, CD4−, CD8−, CD11C−, IgD−, IgM−, CD19+, IgG+. The frequencies of HCoV S-protein-specific IgG memory B cells were as follows; SARS-CoV-2 S (up to ˜8%-range=˜1.6-8%), HCoV-HKU1 S (up to ˜4.3%-range=˜0.2-4.3%), HCoV-NL63 S (up to ˜0.6%-range=˜0.04-0.6%) protein single positive and SARS-CoV-2/HCoV-HKU1 S (up to ˜2.4%-range=˜0.02-2.4%) and SARS-CoV-2/HCoV-NL63 S-protein (up to ˜0.09%-range=˜0-0.09%) double positives. SARS-CoV-2 infected donors showed the presence of SARS-CoV-2/HCoV-HKU1 S-protein cross-reactive IgG memory B cells. Scatter dot plots show frequencies of S protein specific B cells with a line at mean with SD. All differences between means with p-values for each comparison are indicated. **p<0.01; ***p<0.001. A Mann-Whitney two tailed test was used to compare the data groups. Panel c. Pie plots showing immunoglobulin heavy chain distribution of mAbs isolated from 4 COVID donors, CC9, CC10, CC36 and CC40. The majority of the mAbs were encoded by the IgVH3 immunoglobulin gene family. Panel d. Plots showing % nucleotide mutations in heavy (VH) and light (VL) chains of isolated mAbs across different individuals. The VH and VL mutations ranged from 0-11.6% and 0-4.4%, respectively and are shown as scatter dot plots with a line at median. Panel e. CELISA binding curves of isolated mAbs from 4 COVID donors with SARS-CoV-2 and HCoV-HKU1 spikes expressed on 293T cells. Binding to HCoV spikes is recorded as % positive cells using a flow cytometry method. 5 mAbs, 3 from the CC9 donor and 2 from the CC40 donor show cross-reactive binding to SARS-CoV-2 and HCoV-HKU1 spikes. Panel f. Neutralization of SARS-CoV-2 by mAbs isolated from COVID donors. 4 mAbs, 2 each from donors, CC36 and CC40, show neutralization of SARS-CoV-2. The neutralization experiments were performed in duplicate and repeated independently 1-2 times for reproducibility.

FIG. 4. Binding and ADE of SARS-CoV-2/HCoV-HKU1 S-protein specific cross-reactive mAbs. Panel a. Heatmap showing CELISA binding of COVID mAbs to 7 HCoV spikes. Binding represented as area-under-the-curve (AUC) is derived from CELISA binding titrations of mAbs with cell surface-expressed HCoV spikes and the extent of binding is indicated. 5 mAbs show cross-reactive binding across b-HCoV spikes. Panel b. BLI of SARS-CoV-2 and HCoV-HKU1 S-protein-specific cross-reactive mAbs. BLI binding of both IgG and Fab versions of 3 cross-reactive mAbs (CC9.2, CC9.3 and CC40.8) to SARS-CoV-2 and HCoV-HKU1 S-proteins was tested and the binding curves show association (120 s; 180-300) and dissociation rates (240 s; 300-540). BLI binding of antibody-S-protein combinations shows more stable binding (higher binding constants (KDs)) of cross-reactive mAbs HCoV-HKU1 compared to the SARS-CoV-2 S protein. Panel c. Antibody Dependent Enhancement (ADE) activities of cross-reactive mAbs, CC9.2, CC9.3 and CC40.8 binding to SARS-CoV-2 live virus using FcγRIIa (K562) and FcγRIIb (Daudi)-expressing target cells. A dengue antibody, DEN3, was used as a control. Each data point in the curve is derived from the ADE experiment of mAbs with SARS-CoV-2 virus and shows virus titer obtained from technical replicates (n=2); data representative of two independent experiments.

FIG. 5. Epitope specificities of SARS-CoV-2 and endemic HCoV S-protein specific cross-reactive mAbs. Panels a-b. Organization of SARS-CoV-2 S protein subunits, domains and subdomains (Panel a). Epitope mapping of the mAbs binding to domains and subdomains of SARS-CoV-2 S-protein, NTD, RBD, RBD-SD1 and RBD-SD1-2 and heatmap showing BLI responses for each protein. The extent of binding responses is indicated (Panel b). 5 mAbs were specific for RBD, 2 for NTD and the remaining mAbs displayed binding only to the whole S protein. c-d. Negative stain electron microscopy of HCoV-HKU1 S-protein+Fab CC40.8 complex and comparison to MERS-CoV S+Fab G4 complex. (Panel c) Raw micrograph of HCoV-HKU1 S in complex with Fab CC40.8. The Fab-HCoV-HKU1 S protein complexing was performed twice, and the data is representative of the two experiments. (Panel d) Select reference-free 2D class averages with Fabs CC40.8 and Fab G4, which in 2D appear to bind a proximal epitope at the base of the trimer. 2D projections for MERS-CoV S-protein in complex with Fab G4 were generated in EMAN2 from PDB 5W9J.

FIG. 6. Binding of COVID and pre-pandemic sera with HCoV S proteins and neutralization of SARS-CoV-2 virus by COVID sera. Binding of COVID and pre-pandemic sera to cell surface expressed HCoV spikes (CELISA) and soluble S proteins (ELISA). Binding is represented as area-under-the-curve (AUC) and was calculated for each binding titration curve. Binding of sera to non-spike vector only plasmid transfected 293T cells (cell control-CELISA) and to the BSA (ELISA) served as control for non-specific binding. SARS-CoV-2 virus specific ID50 neutralization titers of COVID sera. VSV-g virus served as control for the neutralization assay.

FIG. 7. Correlation of SARS-CoV-2 S binding with virus neutralization. Panel a. Correlation of COVID sera binding to 293T cell surface expressed SARS-CoV-2 spike (CELISA) with SARS-CoV-2 S protein ELISA binding. Binding titers were compared by nonparametric Spearman correlation two-tailed test with 95% confidence interval. The Spearman correlation coefficient (r) and the p-value is indicated. Panel b. Correlation of SARS-CoV-2 ID50 neutralization by COVID sera with CELISA and ELISA S protein binding. ID50 Neutralization titers of sera and CELISA (left) and ELISA (right) S protein binding titers were compared by nonparametric Spearman correlation two-tailed test with 95% confidence interval. The Spearman correlation coefficient (r) and the p-value is indicated.

FIG. 8. Endemic HCoV S protein specific antibody titers in COVID and Pre-pandemic human sera. Panel a. Comparison of endemic HCoV S protein (b-HCoV: HCoV-HKU1 and HCoV-OC43 and α-HCoV: HCoV-NL63 and HCoV-229E) specific CELISA antibody binding titers between SARS-CoV-2 infected (COVID: n=36) and non-infected healthy (Neg: n=36) donors. COVID sera showed higher levels of antibody titers against b-HCoVs, HKU1-CoV and OC43-CoV compared to healthy sera but the antibody levels between the two groups were comparable for binding to α-HCoV S proteins, HCoV-NL63 and HCoV-229E. Statistical comparisons between two groups were performed using Mann-Whitney two tailed test, (**p<0.01; ns-p>0.05). p=0.006 for COVID vs Neg sera for binding to HCoV-HKU1 spike. Panel b. Comparison of endemic HCoV S protein specific CELISA antibody binding titers in COVID donors, with SARS-CoV-2 S specific high (n=18) and low (n=18) antibody binding titers. The COVID donors with higher levels of SARS-CoV-2 S specific antibody titers display significantly higher binding with endemic b-HCoV, HCoV-HKU1 and HCoV-OC43 S proteins compared to individuals with lower SARS-CoV-2 S specific antibody titers. No significant difference for binding to α-HCoV S proteins, HCoV-NL63 and HCoV-229E between the two groups. Statistical comparisons between two groups were performed using Mann-Whitney two tailed test, (**p<0.01; ***p<0.001, ****p<0.0001; ns-p>0.05).

FIG. 9. Further characterization of binding activities of COVID and pre-pandemic sera. Panel a. BioLayer Interferometry (BLI) binding curves of COVID and pre-pandemic sera with SARS-CoV-2, HCoV-HKU1 and HCoV-NL63 S proteins. Panel b. Binding responses and antibody binding off-rates (k_{off}) of COVID/pre-pandemic sera with SARS-CoV-2, HCoV-HKU1 and HCoV-NL63 S proteins.

FIG. 10. Flow cytometry IgG+memory B cell profiling of select COVID19 donors with SARS-CoV-2 S and endemic HCoV-HKU1/HCoV-NL63 S-protein probes. Flow cytometry analysis of the 8 COVID19 donor IgG+ memory B cells specific to SARS-CoV-2 S and endemic b-HCoV, HCoV-HKU1 and α-HCoV, HCoV-NL63 S-protein. The B cells were gated as SSL, CD4−, CD8−, CD11C−, IgD−, IgM−, CD19+, IgG+. The frequencies of HCoV S-protein-specific IgG memory B cells are indicted for each individual. Flow cytometry profiling of the PBMC samples was performed in 3 independent sample batches, as follows, (CC9, CC25, CC28 and CC40), (CC11, CC27 and CC36) and (CC10).

FIG. 11. Immunogenetic properties of the SARS-CoV-2 and HCoV-HKU1 S protein specific mAbs isolated from COVID donors, CC9, CC10, CC36 and CC40.

FIG. 12. ELISA binding, BLI binding and SARS-CoV-2/SARS-CoV-2 virus neutralization of mAbs isolated from 4 COVID donors, CC9, CC10, CC36 and CC40.

### DETAILED DESCRIPTION

### I. Overview

The invention is predicated in part on the studies undertaken by the present inventors to examine cross-reactive serum and memory B cell responses to spike protein in SARS-CoV-2 and endemic coronavirus infection. As detailed herein, the inventors employed a range of immune monitoring assays to compare the serum and memory B cell responses to the S-protein from all 7 coronaviruses infecting humans in SARS-CoV-2 donors and in pre-pandemic donors. It was found that serum cross-reactivity among coronaviruses exists in SARS-CoV-2 convalescent individuals. Specifically, it was observed that the reactivities of IgG+ memory B cells in select COVID-19 donors exhibited differential binding to HCoV spikes including SARS-CoV-2, HCoV-HKU1 (β-HCoV) and HCoV-NL63 (α-HCoV). Additionally, the inventors isolated 5 monoclonal antibodies (mAbs) that exhibited cross-reactive binding with the S proteins of SARS-CoV-2, SARS-CoV-1, MERS-CoV, HCoV-HKU1 and HCoV-OC43. Further, it was demonstrated that cross-reactive mAbs largely target the more conserved S2 subunit on S-proteins, indicative of a SARS-CoV-2 cross-neutralizing epitope on the S2 subunit.

In accordance with these studies, the invention provides specific antibodies that are cross-reactive with the spike protein from multiple coronaviruses. The cross-reactive mAbs that target conserved region between different HCoVs and potentially the emerging SARS-like coronaviruses can have various advantageous utilities. They can be readily used for diagnostics, antibody-based intervention and prophylactic vaccine strategies. They also provide novel tools for studying neutralizing epitopes, e.g., the epitope in the S2 subunit targeted by the CC40.8 cross-neutralizing antibody exemplified herein, that can especially facilitate vaccine design and antibody-based intervention pan-coronavirus vaccine strategies. The invention also provides methods of using such cross-reactive antibodies in the diagnosis, treatment or prevention of coronavirus infections.

The invention can employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al, ed. (1989) Molecular Cloning A Laboratory Manual (2nd ed.; Cold Spring Harbor Laboratory Press); Sambrook et al, ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al, eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al, eds. (1995) Protein Engineering, A Practical Approach (IRL Press at Oxford Univ. Press, Oxford, Eng.). General principles of antibodies and antibody—hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York; Stites et al, eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al, eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al, (Elsevier, Amsterdam); Goldsby et al, eds. (2000) Kuby Immunology (4th ed.; W.H. Freeman & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall, 2003); Harlow and Lane (1988) Antibodies: A Laboratory Manual (Cold Spring Harbor Press); Dieffenbach and Dveksler (2003) PCR Primer (Cold Spring Harbor Press).

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “antibody” also synonymously called “immunoglobulins” (Ig), or “antigen-binding fragment” refers to polypeptide chain(s) which exhibit a strong monovalent, bivalent or polyvalent binding to a given antigen, epitope or epitopes. Unless otherwise noted, antibodies or antigen-binding fragments used in the invention can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments and other designer antibodies that are described below or well known in the art (see, e.g., Serafini, J Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (VH) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4} Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

An antibody-based binding protein, as used herein, may represent any protein that contains at least one antibody-derived V_{H}, V_{L}, or C_{H} immunoglobulin domain in the context of other non-immunoglobulin, or non-antibody derived components. The antibody-based binding proteins of the invention include, but are not limited to (i) Fₑ-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

“Binding affinity” is generally expressed in terms of equilibrium association or dissociation constants (K_{A} or K_{D}, respectively), which are in turn reciprocal ratios of dissociation and association rate constants (k_{off} and kₒₙ, respectively). Thus, equivalent affinities may correspond to different rate constants, so long as the ratio of the rate constants remains the same. The binding affinity of an antibody is usually be expressed as the K_{D} of a monovalent fragment (e.g. a F_{ab} fragment) of the antibody, with K_{D} values in the single-digit nanomolar range or below (subnanomolar or picomolar) being considered as very high and of therapeutic and diagnostic relevance.

As used herein, the term “binding specificity” refers to the selective affinity of one molecule for another such as the binding of antibodies to antigens (or an epitope or antigenic determinant thereof), receptors to ligands, and enzymes to substrates. Thus, all monoclonal antibodies that bind to a particular antigenic determinant of an entity (e.g., a specific epitope of SARS-CoV-2 spike) are deemed to have the same binding specificity for that entity.

A “conservative substitution” with respect to proteins or polypeptides refers to replacement of one amino acid with another amino acid having a similar side chain. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Methods of identifying nucleotide and amino acid conservative substitutions which do not eliminate protein activity are well-known in the art (see, e.g., Brummell et ah, Biochem. 32: 1180-1 187 (1993); Kobayashi et ah, Protein Eng. 12(10):879-884 (1999); and Burks et al, Proc. Natl. Acad. Sci. USA 94: 412-417 (1997)).

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or chemical compounds), combining agents and cells or biological samples, or combining two populations of different cells. Contacting can occur in vitro, e.g., mixing an antibody and a biological sample, or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

A “humanized antibody” is an antibody or antibody fragment, antigen-binding fragment, or antibody-based binding protein comprising antibody V_{H} or V_{L} domains with a homology to human V_{H} or V_{L} antibody framework sequences having a T20 score of greater than 80, as defined by defined by Gao et al. (2013) BMC Biotechnol. 13, pp. 55.

Human coronaviruses (HCoVs) as used herein refer to viruses in the Orthocoronavirinae subfamily of the Coronaviridae virus family. Seven HCoVs have been so far identified, namely HCoV-229E, HCoV-OC43, HCoV-NL63, HCoV-HKU1, severe acute respiratory syndrome coronavirus (SARS-CoV), Middle East respiratory syndrome coronavirus (MERS-CoV) and the novel coronavirus SARS-CoV-2 (aka “2019-nCoV”). Unlike the highly pathogenic SARS-CoV, MERS-CoV, and SARS-CoV-2, the four so-called endemic (or “common”) HCoVs generally cause mild upper-respiratory tract illness and contribute to 15%-30% of cases of common colds in human adults, although severe and life-threatening lower respiratory tract infections can sometimes occur in infants, elderly people, or immunocompromised patients.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, Madison, WI); or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

The term “subject” refers to human and non-human animals (especially non-human mammals). The term “subject” is used herein, for example, in connection with therapeutic and diagnostic methods, to refer to human or animal subjects. Animal subjects include, but are not limited to, animal models, such as, mammalian models of conditions or disorders associated with coronavirus infections. Other specific examples of non-human subjects include, e.g., cows, horses, sheep, pigs, cats, dogs, mice, rats, rabbits, guinea pigs, monkeys.

The terms “treat,” “treating,” “treatment,” and “therapeutically effective” used herein do not necessarily imply 100% or complete treatment. Rather, there are varying degrees of treatment recognized by one of ordinary skill in the art as having a potential benefit or therapeutic effect. In this respect, the inventive method can provide any amount of any level of treatment. Furthermore, the treatment provided by the inventive method can include the treatment of one or more conditions or symptoms of the disease being treated.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

III. SARS-CoV-2 Antibodies with Cross-Reactivity with Other HCoVs

In one aspect, the invention provides novel SARS-CoV-2 antibodies, antigen-binding fragments (aka antibody fragments) thereof, and related antibody-based binding proteins that are cross-reactive with other HCoVs. As exemplified herein, the inventors identified several antibodies that possess such cross-reactivities. The amino acid sequences of the heavy chain and light chain variable regions of these specific antibodies are shown in Table 1. CDR sequences of the heavy chain and light chain of each of the exemplified antibodies are also indicated in Table 1. As detailed in the Examples below, some of these antibodies were also confirmed to have cross-neutralizing activities for SARS-CoV-2 and one or more of the other HCoVs, e.g., antibody CC40.8.

TABLE 1
Variable region sequences of exemplified cross-reactive SARS-COV-2
antibodies (CDRs in each sequence are underlined)

| | | SEQ | CDR |
|---|---|---|---|
| Ab chain | Sequence (SEQ ID NO) | ID NO: | SEQ ID NO: |
| CC40.8_HC | EVQLLESGGGLVQPGGSLRLSCAASGFTFSSYVMTWARQAP | 1 | 10, 11, 12 |
| | GKGLEWVSAISGTGYTYYADSVKGRFTVSRDNSKNTLFLQ | | |
| | MSSLRAEDTAVYYCAITMAPVVWGQGTTVTVSS | | |
| CC40.8_LC | SYELTQPPSVSVSPGQTARITCSGDALPKRYAYWYQQKSGQ | 2 | 13, EDK, 14 |
| | APILVIYEDKKRPSGIPERLSGSKSGTVATLTISGAQVEDEAD | | |
| | YYCYSTDSSGNHAVFGGGTQLTVL | | |
| CC40.5_HC | EVQLVESGGGVVQPGRSLRLSCVASGFTFSNYGMHWVRQA | 3 | 15, 16, 17 |
| | PGKGLEWVAVMSYDGSVTYYGDSVRGRFTISRDNSKNTLY | | |
| | LQMSSLRTDDTGVYYCAKGQPLDDIWGLGTLVTVSS | | |
| CC40.5_LC | EIVMTQSPLSLPVSLGQSASISCRSSQSLVHTDGITYLSWFQQ | 4 | 18, KVS, 19 |
| | RPGQSPRRLLYKVSNRDSGVPDRFSGSGSGTDFTLKISRVEA | | |
| | EDVGVYYCLQGTYWPWTFGQGTKVEIK | | |
| CC9.1/2_HC | EVQLVESGGGLVQPGRSLRLSCVAPGFTLGGYGMHWVRQA | 5 | 20, 21, 22 |
| | PGKGLEWVGLISYDGSVQKYGSSVKGRFTISKDNSKNTLYL | | |
| | EMNSLRADDTAVYFCVKGASLGDNWGQGTLVTVSS | | |
| CC9.1_LC | EIVMTQSPLSLPVTLGQPASISCRSSQSLVYSDGDTYMSWFH | 6 | 23, KVS, 24 |
| | QRPGQSPRRLIYKVSNRDSGVPDRFSGSGSGTDFTLKISRVE | | |
| | AEDVGVYYCMQGTHWPYTFGPGTKVEIK | | |
| CC9.2_LC | EIVMTQSPLSLPVTLGQPASISCRSSQSLVYSDGDTYMSWFH | 7 | 23, KVS, 24 |
| | QRPGQSPRRLIYKVSNRDFGVPDRFSGSGSGTDFTLKISRVE | | |
| | AEDVGVYYCMQGTHWPYTFGPGTKVEIK | | |
| CC9.3_HC | QVQLQESGPGLVKPSETLSLTCTVSGGSIRSDFWSWIRQFPG | 8 | 25, 26, 27 |
| | KGLEWIGYIYYSGSTNYNPSLESRVTISVDTSKNEFSLKLNY | | |
| | VTAADTAIYYCARETRWNWLDSWGQGTLVTVSS | | |
| CC9.3_LC | DIVMTQSPDSLAVSLGERASINCKSSQTISYISNNKNYLAWY | 9 | 28, WAS, 29 |
| | QQKPGQPPRLLIYWASTRESGVPDRFSGGGFGTDFTLTISSL | | |
| | QAEDVAVYYCQQYFNTPWTFGQGTKVEIK | | |

The antibodies or antigen-binding fragments of the invention are derived from one of the exemplified antibodies shown in the Table 1. Typically, they have identical or substantially identical heavy chain and light chain CDR sequences as that of one of the exemplified antibodies. Defined alternatively, they have the same binding specificity as that of one of the exemplified antibodies. In some embodiments, the antibodies or antigen-binding fragments have heavy chain and light CDR sequences that are respectively identical to the heavy chain and light chain CDR sequences of one of the antibodies listed in Table 1. In some embodiments, the antibodies or antigen-binding fragments have heavy chain and light CDR sequences that are substantially identical, respectively, to the heavy chain and light chain CDR sequences of one of the antibodies listed in Table 1. For example, the antibodies can have heavy chain CDR sequences (HCDR1-s) and light chain CDR sequences (LCDR1-s) that are respectively identical to SEQ ID NOs:10-12, EDK, and SEQ ID NO:14, except for one or more conservative substitutions. Other than the identical or substantially identical CDR sequences, the antibodies or antigen-binding fragments can have a heavy chain variable region sequence and a light chain variable region sequence that are substantially identical (e.g., about 95%, 96%, 97%, 98%, or 99% identical) to the heavy chain and light variable region sequences of one of antibodies listed in Table 1, respectively.

In some embodiments, the antibodies or antigen-binding fragments of the invention have the same binding specificity as that of CC40.8, which has heavy and light chain variable region sequences respectively shown as SEQ ID NO:1 and 2. In some of these embodiments, the antibodies or antigen-binding fragments of the invention have heavy chain CDR1-3 sequences that are respectively identical to SEQ ID NOs:10-12, and light chain CDR1-3 sequences that are respectively identical to SEQ ID NO:13, EDK, and SEQ ID NO:14. Other than the CDR sequences, the heavy chain and light chain variable regions of these antibodies or antigen-binding fragments can each have one or more amino acid substitutions in the framework regions relative to SEQ ID NO:1 and 2, respectively. In various embodiments, the amino acid substitutions can be either conservative or non-conservative substitutions. In some of these embodiments, at least one of the amino acid substitutions in the heavy chain and/or light chain framework region is conservative substitution. In some embodiments, all of the amino acid substitutions in the heavy chain and/or light chain framework region are conservative substitutions. In some of the embodiments, at least one of the amino acid substitutions in the heavy chain and/or light chain framework region is non-conservative substitution. In some embodiments, all of the amino acid substitutions in the heavy chain and/or light chain framework region are non-conservative substitutions.

In some embodiments, the antibodies or antigen-binding fragments of the invention have heavy chain CDR1-3 sequences that are respectively identical to SEQ ID NOs:15-17, and light chain CDR1-3 sequences that are respectively identical to SEQ ID NO:18, KVS, and SEQ ID NO:19. In some embodiments, the antibodies or antigen-binding fragments of the invention have heavy chain CDR1-3 sequences that are respectively identical to SEQ ID NOs:20-22, and light chain CDR1-3 sequences that are respectively identical to SEQ ID NO:23, KVS, and SEQ ID NO:24. In some other embodiments, the antibodies or antigen-binding fragments of the invention have heavy chain CDR1-3 sequences that are respectively identical to SEQ ID NOs:25-27, and light chain CDR1-3 sequences that are respectively identical to SEQ ID NO:28, WAS, and SEQ ID NO:29. In various embodiments, the antibodies or antigen-binding fragments of the invention have heavy chain variable region and light chain variable region sequences that are substantially identical (e.g., about 95%, 96%, 97%, 98%, or 99% identical) to SEQ ID NOs:1 and 2, SEQ ID NOs:3 and 4, SEQ ID NOs:5 and 6, SEQ ID NOs:5 and 7, or SEQ ID NOs:8 and 9, respectively. In some of these embodiments, in addition to the substantial sequence identity over the entire heavy chain variable region and light chain variable region, the antibodies or antigen-binding fragments have heavy chain and light chain CDRs that are respectively identical to that of one of the antibodies shown in Table 1 (e.g., antibody 40.8). In some embodiments, the antibodies or antigen-binding fragments of the invention have a heavy chain variable region and a light chain variable region, one or both of which are respectively identical to SEQ ID NOs:1 and 2, SEQ ID NOs:3 and 4, SEQ ID NOs:5 and 6, SEQ ID NOs:5 and 7, or SEQ ID NOs:8 and 9.

Antibodies of the invention include intact antibodies (e.g., IgG1 antibodies exemplified herein), antibody fragments or antigen-binding fragments, antibody-based binding proteins, which contain the antigen-binding portions of an intact antibody that retain capacity to bind to SARS-CoV-2 spike protein and cross-react with the spike protein of one or more of the other HCoVs (e.g., SARS-CoV spike). Examples of such antibody fragments include (i) a F_{ab} fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and CHI domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} or V_{L} domain (see, e.g., Ward et al., Nature 341:544-546, 1989); and (vii) an isolated complementarity determining region (CDR) as a linear or cyclic peptide. Examples of antibody-based binding proteins are polypeptides in which the binding domains of the antibodies are combined with other polypeptides or polypeptide domains, e.g. alternative molecular scaffolds, Fc-regions, other functional or binding domains of other polypeptides or antibodies resulting in molecules with addition binding properties, e.g. bi- or multispecific proteins or antibodies. Such polypeptides can create an arrangement of binding or functional domains normally not found in naturally occurring antibodies or antibody fragments.

Antibodies of the invention also encompass “antibody fragments” (also termed “antigen-binding fragments” herein) that contain portions of an intact IgG antibody (e.g., the variant regions) responsible for target antigen recognition and binding. One example of such antibody fragments is single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules.

Antibodies of the present invention also encompass single domain antigen-binding units, which have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

The various antibodies, antibody-based binding proteins, and antibody fragments thereof described herein can be produced by enzymatic or chemical modification of the intact antibodies, or synthesized de novo using recombinant DNA methodologies, or identified using phage display libraries. Methods for generating these antibodies, antibody-based binding proteins, and antibody fragments thereof are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nat. Struct. Biol. 11:500-515, 2002; Ghahroudi et al., FEBS Letters 414:521-526, 1997; and Bond et al., J. Mol. Biol. 332:643-55, 2003. Other types of antigen-binding fragments (e.g., Fab, F(ab′)₂ or Fd fragments) can also be readily produced with routinely practiced immunology methods. See, e.g., Harlow & Lane, Using Antibodies, A Laboratory Manual, Cold Spring Harbor Laboratory Press, Cold Spring Harbor, New York, 1998.

In some embodiments, an antibody or antigen-binding fragment of the invention can be further conjugated to a synthetic molecule such as a marker or detectable moiety (or label). Recombinant engineering and incorporated selenocysteine (e.g., as described in U.S. Pat. No. 8,916,159) can be used to conjugate a synthetic molecule. Other methods of conjugation can include covalent coupling to native or engineered lysine side-chain amines or cysteine side-chain thiols. See, e.g., Wu et al., Nat. Biotechnol, 23: 1 137-1 146 (2005).

The antibodies or antigen-binding fragments of the invention can be generated in accordance with routinely practiced immunology methods. Some of such methods are exemplified herein in the Examples. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, New York, 1998; Kohler & Milstein, Nature 256:495-497, 1975; Kozbor et al., Immunology Today 4:72, 1983; and Cole et al., pp. 77-96 in Monoclonal Antibodies and Cancer Therapy, 1985.

### IV. Polynucleotides, Vectors and Host Cells for Producing Cross-Reactive Coronavirus Antibodies

The invention provides substantially purified polynucleotides (DNA or RNA) that are identical or complementary to sequences encoding polypeptides comprising segments or domains of the antibody, antibody-based binding protein or antibody fragment thereof chains described herein. In some embodiments, the polynucleotides of the invention encode the heavy chain or light chain sequences of cross-reactive coronavirus antibodies that are derived from one of the exemplified antibodies, e.g., an antibody derived from CC40.8. When expressed from appropriate expression vectors, polypeptides encoded by these polynucleotides are capable of exhibiting coronavirus cross-reactive capacity. Also provided in the invention are polynucleotides which encode at least one CDR region and usually all three CDR regions from the heavy or light chain of the antibodies described herein. Some other polynucleotides encode all or substantially all of the variable region sequence of the heavy chain and/or the light chain of the exemplified antibodies. For example, some of these polynucleotides encode the amino acid sequence of the heavy chain variable region shown in SEQ ID NO:1, and/or the amino acid sequence of the light chain variable region shown in SEQ ID NO:2. Because of the degeneracy of the code, a variety of nucleic acid sequences will encode each of the immunoglobulin amino acid sequences.

The polynucleotides of the invention can encode only the variable region sequences of the exemplified antibodies. They can also encode both a variable region and a constant region of the antibody. Some of polynucleotide sequences of the invention nucleic acids encode a mature heavy chain variable region sequence that is substantially identical (e.g., at least 80%, 90%, 95% or 99%) to the mature heavy chain variable region sequence shown in SEQ ID NO:1. Some other polynucleotide sequences encode a mature light chain variable region sequence that is substantially identical (e.g., at least 80%, 90%, 95% or 99%) to the mature light chain variable region sequence shown in SEQ ID NO:2. Some of the polynucleotide sequences encode a polypeptide that comprises variable regions of the heavy chain or the light chain of one of the exemplified antibodies. Some other polynucleotides encode two polypeptide segments that respectively are substantially identical to the variable regions of the heavy chain or the light chain of one of the exemplified antibodies (e.g., antibody CC40.8).

The polynucleotide sequences can be produced by de novo solid-phase DNA synthesis or by PCR mutagenesis of an existing sequence (e.g., sequences as described in the Examples below) encoding an exemplified functional antibody. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, C A, 1990; Mattila et al., Nucleic Acids Res. 19:967, 1991; and Eckert et al., PCR Methods and Applications 1:17, 1991.

Also provided in the invention are expression vectors and host cells for producing the functional antibodies described herein. Specific examples of plasmid and transposon based vectors for expressing the antibodies are described in the Examples below. Various other expression vectors can also be employed to express the polynucleotides encoding the functional antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the antibody polynucleotides and polypeptides in mammalian (e.g., human) cells include pCEP4, pREP4, pThioHis A, B & C, pcDNA3.1/His, pEBVHis A, B & C (Invitrogen, San Diego, CA), MPSV vectors, and numerous other vectors known in the art for expressing other proteins. Other useful nonviral vectors include vectors that comprise expression cassettes that can be mobilized with Sleeping Beauty, PiggyBack and other transposon systems. Useful viral vectors include vectors based on lentiviruses or other retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a functional antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of a functional antibody chain or fragment. These elements typically include an ATG initiation codon and adjacent ribosome binding site (Kozak consensus sequence) or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted functional antibody sequences. More often, the inserted functional antibody sequences are linked to a signal sequences before inclusion in the vector. Vectors to be used to receive sequences encoding the functional antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human, and preferably of human IgG1 antibodies.

The host cells for harboring and expressing the functional antibody chains can be either prokaryotic or eukaryotic. In some preferred embodiments, mammalian host cells are used to express and to produce the antibody polypeptides of the present invention. For example, they can be either a hybridoma cell line expressing endogenous immunoglobulin genes or a mammalian cell line harboring an exogenous expression vector. These include any normal mortal or normal or abnormal immortal animal or human cell. In addition to the cell lines exemplified herein, a number of other suitable host cell lines capable of secreting intact immunoglobulins are also known in the art. These include, e.g., the CHO cell lines, various HEK 293 cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer, and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, EF1α and human UbC promoters exemplified herein, the metallothionein promoter, the constitutive adenovirus major late promoter, the dexamethasone-inducible MMTV promoter, the SV40 promoter, the MRP pol III promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transformation is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate for the cell type.

The invention further provides eukaryotic or non-eukaryotic cells (e.g., T lymphocytes) that have been recombinantly engineered to produce the antibodies, antibody-based binding proteins or antibody fragments thereof of the invention. The eukaryotic or non-eukaryotic cells can be used as an expression system to produce the antibody of the invention. In some embodiments, the invention provides coronavirus spike targeting immune cells that are engineered to recombinantly express a cross-reactive coronavirus antibody of the invention. For example, the invention provides a T cell engineered to express an antibody of the invention (e.g., an scFv, scFv-Fc, or (scFv)2), which is linked to a synthetic molecule containing one or more of the following domains: a spacer or hinge region (e.g., a CD28 sequence or a IgG4 hinge-Fc sequence), a transmembrane region (e.g., a transmembrane canonical domain), and an intracellular T-cell receptor (TCR) signaling domain, thereby forming a chimeric antigen receptor (CAR) or T-body. Intracellular TCR signaling domains that can be included in a CAR (or T-body) include, but are not limited to, CD3ζ, FcR-γ, and Syk-PT signaling domains as well as the CD28, 4-1BB, and CD134 co-signaling domains. Methods for constructing T-cells expressing a CAR (or T-body) are known in the art. See, e.g., Marcu-Malina et al., Expert Opinion on Biological Therapy, Vol. 9, No. 5 (posted online on Apr. 16, 2009).

### V. Therapeutic and Diagnostic Applications

The cross-reactive coronavirus antibodies or antigen-binding fragments thereof disclosed herein can be used in various therapeutic and diagnostic applications. For example, they can be used alone or in a combination therapy in the prophylactic or therapeutic treatment of coronavirus infections (e.g., SARS-CoV-2 infection). In some embodiments, the invention provides methods of using the cross-reactive coronavirus antibodies or fragments thereof to treat patients having infection by one or more coronaviruses (e.g., SARS-CoV-2 and SARS-CoV) or patients having other diseases or conditions associated with coronavirus infections. In some embodiments, the antibodies or antigen-binding fragments of the invention can be used to prevent infections by one or more coronaviruses, or to reduce or manage coronavirus-induced symptoms in a subject infected with one or more coronaviruses. In some other embodiments, the invention provides diagnostic methods for detecting coronavirus related infections or the presence of coronavirus in biological samples obtained from human subjects.

Pharmaceutical compositions containing one or more of the cross-reactive coronavirus antibodies or antigen-binding fragments described herein are encompassed by the invention. In some embodiments, the pharmaceutical compositions are employed in therapeutic methods for treating coronavirus infections. Typically, the subject or patient suitable for treatment is one who has been or is suspected of having been exposed to one or more coronaviruses (e.g., SARS-CoV-2 or SARS-CoV), is infected or suspected of being infected with one or more coronavirus, has a coronavirus related disease, has a symptom of a coronavirus related disease, or has a predisposition toward contracting a coronavirus related disease. For example, the subject to be treated can be one who has been diagnosed of SARS-CoV-2 infection and/or possess symptoms associated with infections by one or more HCoVs. The cross-reactive coronavirus antibody or antigen-binding fragment thereof for use in the methods of the invention can a human or humanized antibody. In some embodiments, the cross-reactive coronavirus antibody or antigen-binding fragment thereof comprises a binding domain that binds to the same epitope as, or competitively inhibits binding of, one or more of the antibodies exemplified herein. In addition to the antibodies, pharmaceutical compositions typically also contain a pharmaceutically acceptable, non-toxic, sterile carrier such as physiological saline, non-toxic buffers, preservatives and the like.

Therapeutic methods of the invention typically involve administering to a subject in need of treatment a pharmaceutical composition that contains a therapeutically amount of a cross-reactive coronavirus antibody or antigen-binding fragment described herein (e.g., antibody CC40.8 or an antibody derived from CC40.8). A therapeutically effective amount refers to an amount sufficient to achieve a therapeutic benefit, e.g., to ameliorate symptoms associated with HCoV infections. Suitable amount to be administered can be readily determined by one of ordinary skill in the art without undue experimentation given the invention. Factors influencing the mode of administration and the respective amount of a cross-reactive coronavirus neutralizing antibody or antigen-binding fragment thereof include, but are not limited to, the severity of the disease, the history of the disease, and the age, height, weight, health, and physical condition of the individual undergoing therapy. Similarly, the amount of a cross-reactive coronavirus immunotherapeutic to be administered will be dependent upon the mode of administration and whether the subject will undergo a single dose or multiple doses of this agent. In some embodiments, the therapeutic methods of the invention can be employed in combination with other regimen for treating or controlling HCoV infections. These include, e.g., remdesivir, Bamlanivimab, Casirivimab and Imdevimab cocktail, hydroxychloroquine and chloroquine, interferon β-1a, Azithromycin, Tocilizumab and other IL-6 inhibitors, Interferon-γ, or intravenous fluids and balancing electrolytes.

Methods of preparing and administering a cross-reactive coronavirus antibody or antigen-binding fragment thereof provided herein, to a subject in need thereof are well known to or can be readily determined by those skilled in the art. The route of administration of a cross-reactive coronavirus neutralizing antibody or antigen-binding fragment thereof can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as suitable forms, another example of a form for administration would be a solution for injection, in particular for intravenous or intraarterial injection or drip. In some cases a suitable pharmaceutical composition can comprise a buffer (e.g. acetate, phosphate or citrate buffer), a surfactant (e.g. polysorbate), optionally a stabilizer agent (e.g. human albumin), etc. In other methods compatible with the teachings herein, a cross-reactive coronavirus antibody or antigen-binding fragment thereof as provided herein can be delivered directly to a site where the binding molecule can be effective in virus neutralization, e.g., the endosomal region of a coronavirus-infected cell. Preparation of pharmaceutical compositions of the invention and their various routes of administration can be carried out in accordance with methods well known in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978.

The invention also provided methods for using the cross-reactive coronavirus antibodies or related antigen-binding fragments described herein in diagnostic methods for detecting HCoV infections or the presence of HCoVs. Various assays routinely practiced in the art can be employed for performing the diagnostic methods. These include, e.g., competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety). Methods and reagents suitable for determination of binding characteristics of a cross-reactive HCoV antibody or antigen-binding fragment thereof are known in the art and/or are commercially available. Equipment and software designed for such kinetic analyses are commercially available (e.g., BIAcore®, BIAevaluation® software, GE Healthcare; KINEXA® Software, Sapidyne Instruments).

The diagnostic methods of the invention typically involve obtaining a biological sample from a subject that has or is suspected of having been infected with a coronavirus spike. Preferably, the subject is a human. In various embodiments, the biological sample suitable for the assays can be blood or any fraction thereof (e.g., serum, plasma, or whole blood), urine, feces, saliva, vomitus, or any combination thereof. Utilizing the novel antibodies disclosed herein, presence of a coronavirus spike or spike derived antigen in the biological sample can be readily determined with any of the various immunoassays described herein, e.g., ELISA.

The invention further provides kits that contain a cross-reactive coronavirus immunotherapeutic of the invention for performing the therapeutic or diagnostic applications described herein. Typically, the kits contain two or more components required for performing the therapeutic or diagnostic methods of the invention. Kit components include, but are not limited to, one or more the disclosed antibodies or antibody fragments thereof, appropriate reagents, and/or equipment. In some embodiments, the kits can contain an antibody or antibody fragment thereof of the invention and an immunoassay buffer suitable for detecting HCoV spike proteins (e.g. by ELISA, flow cytometry, magnetic sorting, or FACS). The kit may also contain one or more microtiter plates, standards, assay diluents, wash buffers, adhesive plate covers, magnetic beads, magnets, and/or instructions for carrying out a method of the invention using the kit. The kit scan include an antibody or antigen-binding fragment thereof of the invention bound to a substrate (e.g., a multi-well plate or a chip), which is suitably packaged and useful to detect HCoV spike antigens. In some embodiments, the kits include an antibody or antibody fragment thereof of the invention that is conjugated to a label, such as, a fluorescent label, a biologically active enzyme label, a luminescent label, or a chromophore label. The kits can further include reagents for visualizing the conjugated antibody or antibody fragment thereof, e.g., a substrate for the enzyme. some embodiments, the kits include an antibody or antibody fragment thereof of the invention that is conjugated to a contrast agent and, optionally, one or more reagents or pieces of equipment useful for imaging the antibody in a subject.

Generally, the cross-reactive coronavirus antibodies or antibody fragments thereof of the invention in a kit are suitably packaged, e.g., in a vial, pouch, ampoule, and/or any container appropriate for a therapeutic or detection method. Kit components can be provided as concentrates (including lyophilized compositions), which may be further diluted prior to use, or they can be provided at the concentration of use. For use of the antibody of the invention in vivo, single dosages may be provided in sterilized containers having the desired amount and concentration of components.

In various applications, the cross-reactive coronavirus antibodies of the invention can be employed to produce antibody derivatives such as immunoconjugates. In some embodiments, the antibodies of the invention can be linked to a therapeutic moiety, such as a cytotoxin, a drug or a radioisotope. When conjugated to a cytotoxin, these antibody conjugates are referred to as “immunotoxins.” A cytotoxin or cytotoxic agent includes any agent that is detrimental to (e.g., kills) cells. Techniques for conjugating such therapeutic moiety to antibodies are well known in the art. In some embodiments, antibodies of the invention can be conjugated to an appropriate detectable agent to form immunoconjugates for use in diagnostic applications and in vivo imaging. The detectable agents can be any chemical moieties that contain a detectable label, e.g., radioisotopes, enzymes, fluorescent labels and various other antibody tags. In some other embodiments, the cross-reactive coronavirus antibodies of the invention can be further modified to contain additional non-proteinaceous moieties that are known in the art and readily available. The moieties suitable for derivatization of the antibody include but are not limited to water soluble polymers, e.g., polyethylene glycol (PEG).

### EXAMPLES

The following examples are provided to further illustrate the invention but not to limit its scope. Other variants of the invention will be readily apparent to one of ordinary skill in the art and are encompassed by the appended claims.

### Example 1. Serum Cross-Reactivity to S Protein in COVID-19 and Pre-Pandemic Donors

Since individuals who have been infected with SARS-CoV-2 will generally also have been infected with endemic HCoVs, we chose to compare COVID-19 and pre-pandemic donors in terms of serum Abs and BCRs with specificity for the spike (S) protein (Table 2 summarizes the demographic details of the human cohorts). The rationale was that the pre-pandemic donor cross-reactive responses could only be due to endemic HCoV infection. However, the COVID-19 cohort could reveal the effects of SARS-CoV-2 infection on cross-reactive responses.

TABLE 2
Demographic information of COVID-19 and pre-pandemic
HIV seropositive healthy human cohorts.

| | COVID donor | | Pre-pandemic donors | |
|---|---|---|---|---|
| | (n = 36) | | (n = 36) | |
| Age (years) | 20-72 | (median = 48) | 23-79 | (median = 43) |
| Gender | | | | |
| Male | 50% | (18/36) | 83% | (30/36) |
| Female | 50% | (18/36) | 17% | (6/36) |
| Race/Ethnicity | | | | |
| White, non-Hispanic | 78% | (28/36) | 56% | (20/36) |
| Hispanic | 11% | (4/36) | 33% | (12/36) |
| Black, non-Hispanic | 0% | (0/36) | 8% | (3/36) |
| Asian, non-Hispanic | 5.5% | (2/36) | 3% | (1/36) |
| Unknown | 5.5% | (2/36) | 0% | (0/36) |

| | | | |
|---|---|---|---|
| SARS-CoV-2 PCR | 81% | (29/36) | N/A |
| Positivity | | | |
| Lateral Flow Positivity | 90% | (25/36) | N/A |
| Disease Severity | | | |
| Mild | 58% | (21/36) | N/A |
| Mild to Moderate | 5.5% | (2/36) | N/A |
| Moderate | 17% | (6/36) | N/A |
| Moderate to Severe | 11% | (4/36) | N/A |
| Severe | 5.5% | (2/36) | N/A |
| Critical | 3% | (1/36) | N/A |
| Symptoms | | | |
| Cough | 78% | (28/36) | N/A |
| Fever | 58% | (21/36) | N/A |
| Fatigue | 28% | (10/36) | N/A |
| Anosmia | 25% | (9/36) | N/A |
| Dyspnea | 25% | (9/36) | N/A |
| Diarrhea | 11% | (4/36) | N/A |
| Days Post Symptom | 6-67 | (median = 30) | N/A |
| Onset at Collection | | | |

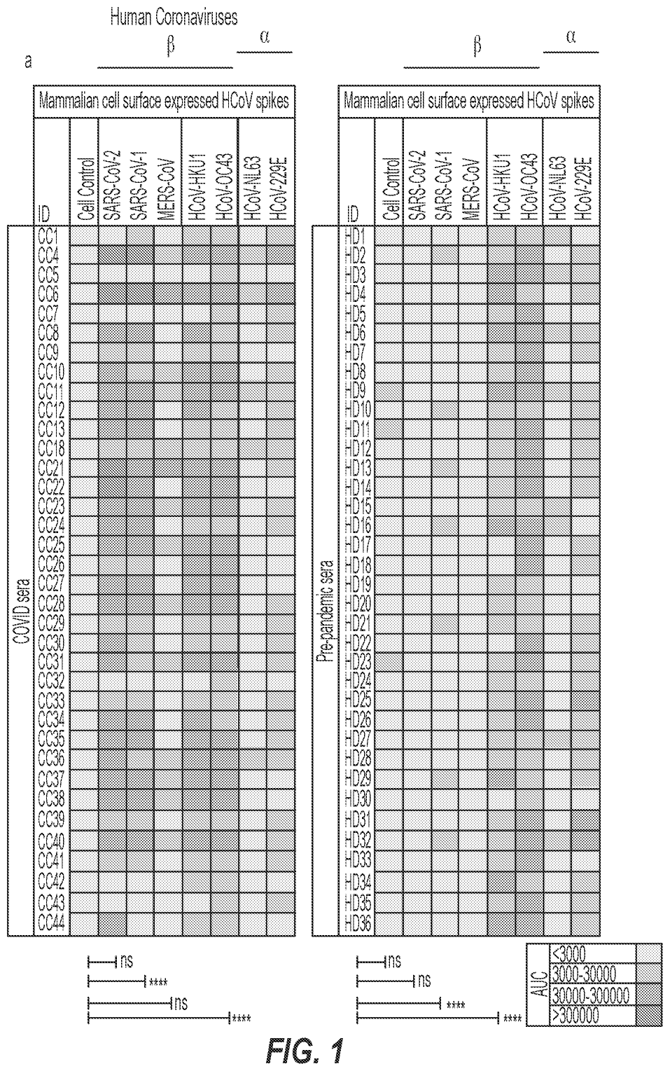

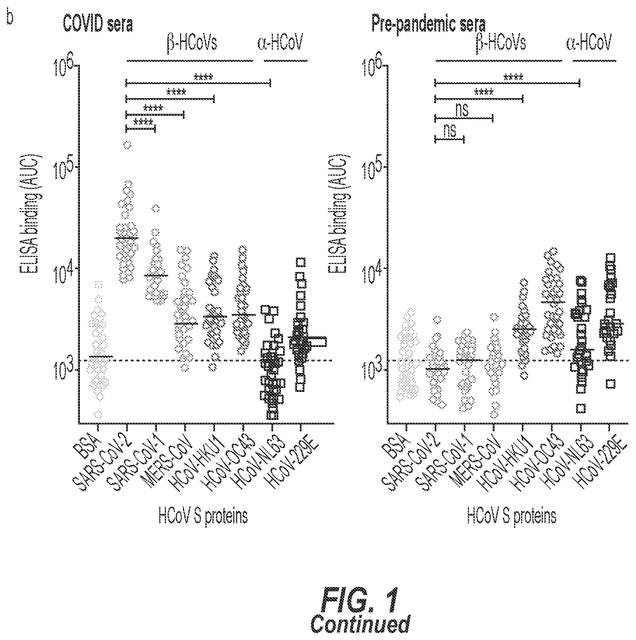

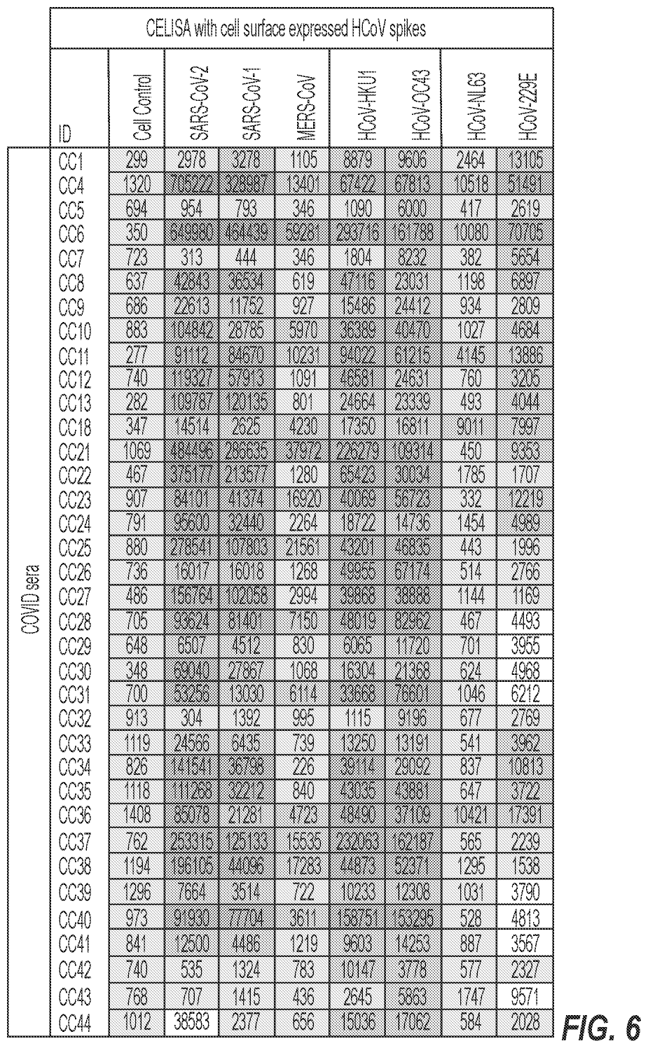

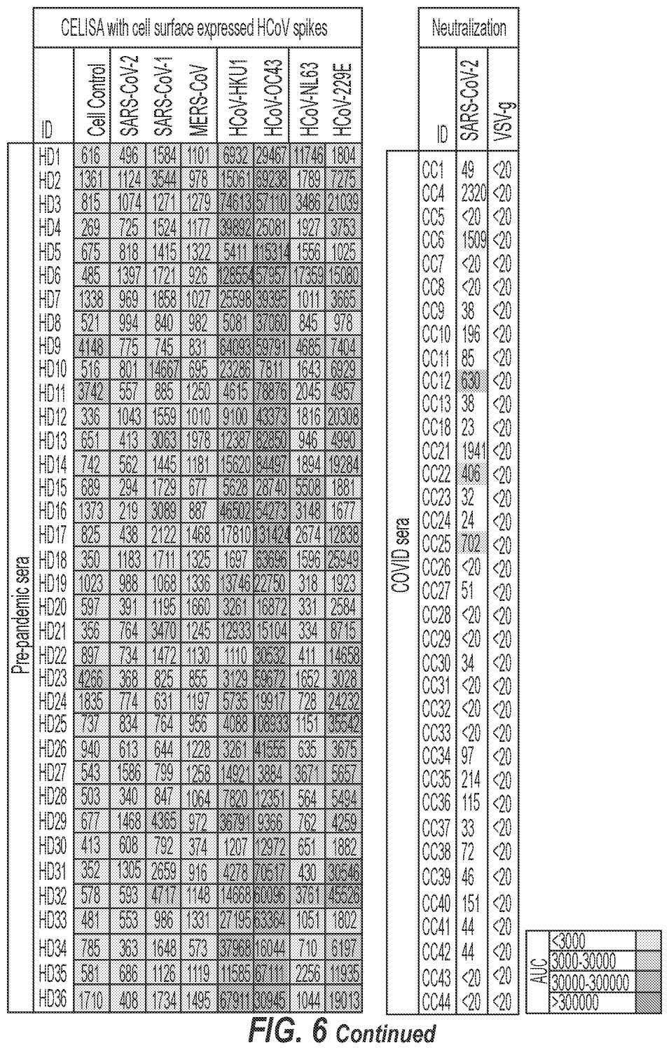

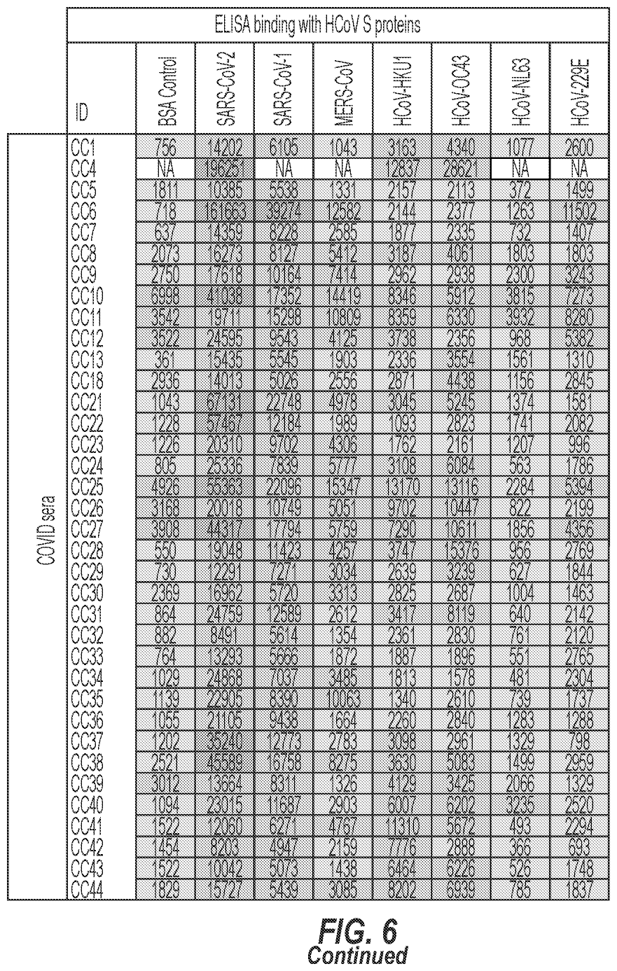

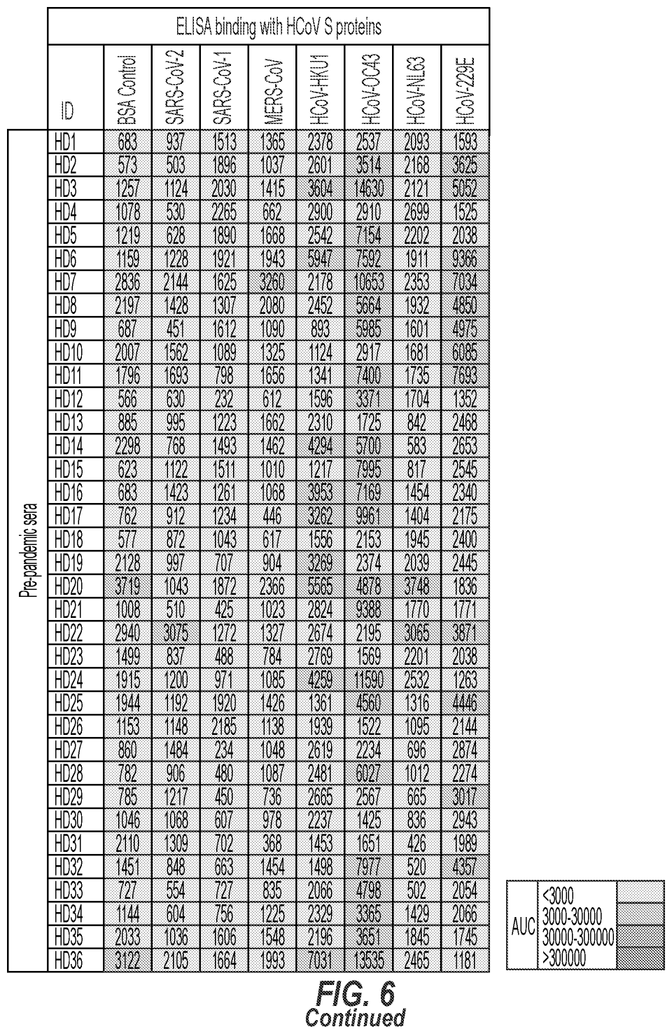

To assess serum Ab S-protein binding in the two cohorts, we used cell-surface and recombinant soluble S proteins. We employed both binding assays in parallel to assess any potential differences in the serum antibody binding patterns that may result from the engineering and truncation of the soluble HCoV S protein relative to the membrane-bound protein. HIV envelope studies have revealed that the cell surface expressed envelope trimers may more closely mimic infectious virion-associated envelope spikes, including in terms of native-like glycan compositions. First, we developed and utilized a high-throughput flow cytometry-based cell surface spike binding assay (Cell-based ELISA; CELISA). COVID-19 convalescent sera from 36 donors showed strong reactivity to the SARS-CoV-2 spike in the vast majority of infected donors (FIG. 1a, FIG. 6), somewhat lower reactivity with the SARS-CoV-1 spike and much lower reactivity with the MERS-CoV spike in a pattern consistent with sequence conservation between the 3 viruses. COVID sera also exhibited strong cross-reactivity with endemic HCoV spikes, especially with the HCoV-HKU1 and HCoV-OC43 b-HCoVs (FIG. 1a). The α-HCoV- derived HCoV-NL63 spike was least reactive among the 4 endemic HCoVs. Next, we tested sera from a cohort of 36 HIV seropositive but otherwise healthy human donors whose samples were collected pre-pandemic. The sera showed minimal or no reactivity to SARS-CoV-2/CoV-1 and MERS-CoV spikes but showed strong binding to the endemic HCoV spikes, especially against the HCoV-HKU1 and HCoV-OC43 b-HCoVs (FIG. 1, FIG. 6). The results suggest that the pre-pandemic sera, at least in our cohort, possess low levels of pre-existing SARS-CoV-2 circulating Abs.

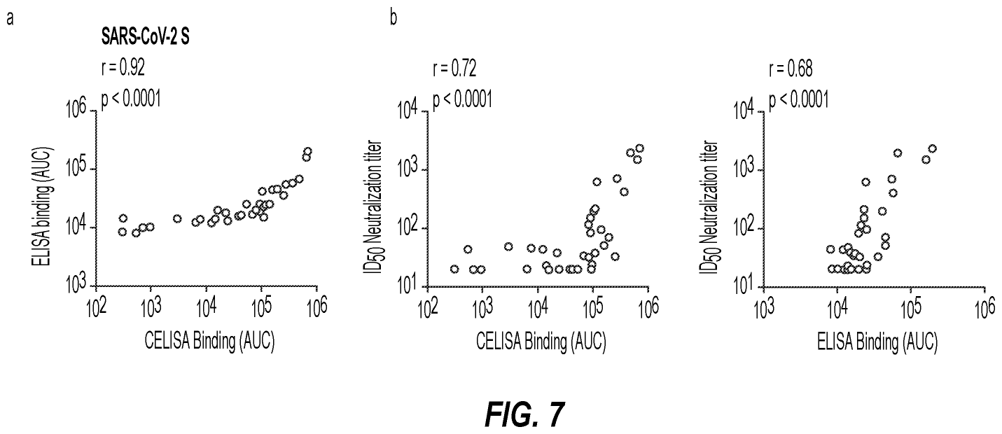

To further investigate, we generated recombinant soluble S proteins of all 7 HCoVs using a general stabilization strategy described elsewhere Kirchdoerfer et al., Nature 531, 118-121, 2016; Pallesen et al., Proc. Natl. Acad. Sci. USA 114, E7348-E7357, 2017; and Wrapp et al., Science 367, 1260-1263, 2020. ELISA showed a similar binding pattern of the COVID and pre-pandemic sera as the CELISA (FIG. 1B, FIG. 6). The SARS-CoV-2 S specific binding of COVID sera in the two assay formats (CELISA versus ELISA) correlated strongly (r=0.92, p<0.001) (FIG. 7), the titers detected in ELISA being substantially lower overall. Differential sensitivity of the two assay formats may reflect an inherently greater sensitivity of flow cytometry (CELISA) compared to ELISA but also to the potential effects of engineering on the soluble HCoV S proteins that may reduce nativity of some epitopes. We also tested the neutralization of the COVID sera with SARS-CoV-2 and the ID₅₀ neutralization titers positively correlated with both binding assays (CELISA (r=0.72, p<0.0001), ELISA (r=0.68, p<0.0001)) (FIG. 7). Overall, both CELISA and ELISA revealed binding Abs to all 7 HCoV spikes in COVID sera but only to endemic HCoVs in the pre-pandemic sera.

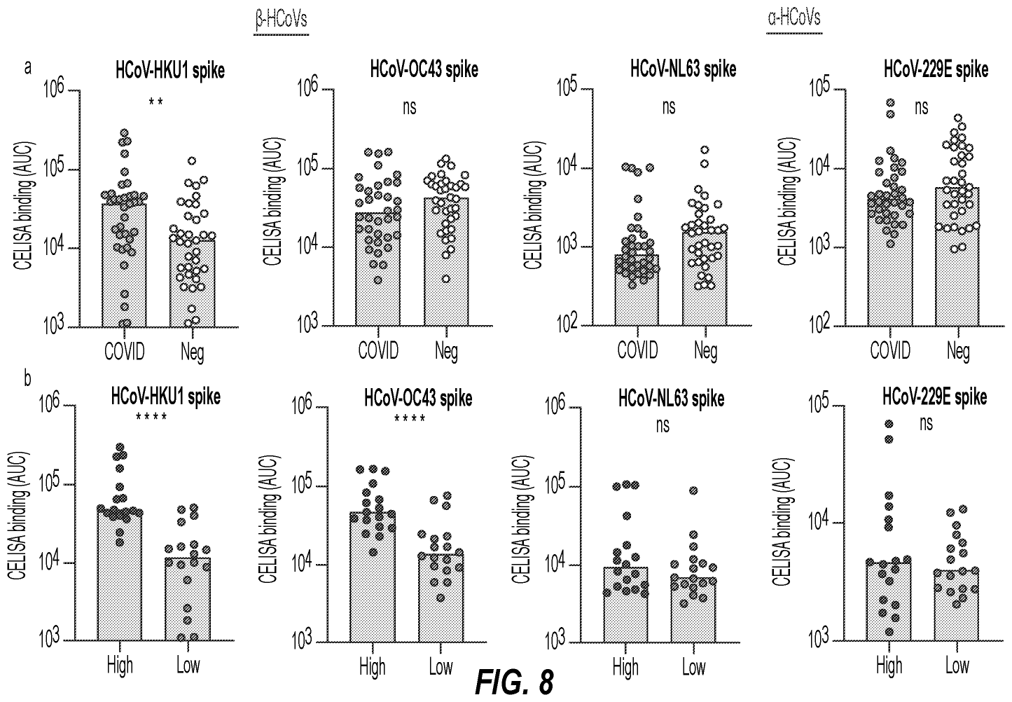

To assess whether SARS-CoV-2 infection may impact serum Ab titers to endemic HCoVs, we compared Ab titers to endemic HCoV S-protein in sera from COVID and pre-pandemic cohorts. Higher CELISA Ab titers to endemic HCoV-HKU1 S-protein, but not for other HCoV spikes (HCoV-OC43, HCoV-NL63 and HCoV-229E) were observed in the COVID cohort compared to the pre-pandemic cohort (FIG. 8). The result suggests that SARS-CoV-2 infection may boost titers to the related HCoV-HKU1 spike (Chen et al., J Med Virol 92, 418-423, 2020; and Lu et al. Lancet 395, 565-574, 2020). To further investigate, we divided individuals from the COVID cohort into two groups, one with the higher SARS-CoV-2 spike Ab titers (AUC>85,000) and the other with lower titers (AUC<85,000). Consistent with the above result, the COVID sera with higher SARS-CoV-2 titers showed significantly higher binding to HCoV-HKU1 and HCoV-OC43 S-proteins compared to the low titer group (FIG. 8). The α-HCoVs HCoV-NL63 and HCoV-229E spike binding antibody titers were comparable between the two groups and served as a control (FIG. 8). Since the two cohorts are not matched in terms of a number of parameters and are of limited size, any conclusions should be treated with caution. Nevertheless, it is noteworthy that SARS-CoV-2 infection is apparently associated with enhanced b-HCoVs S-protein Ab responses. A key question is whether the enhanced responses arise from de novo B cell responses or from a recall response of B cells originally activated by an endemic HCoV virus infection.

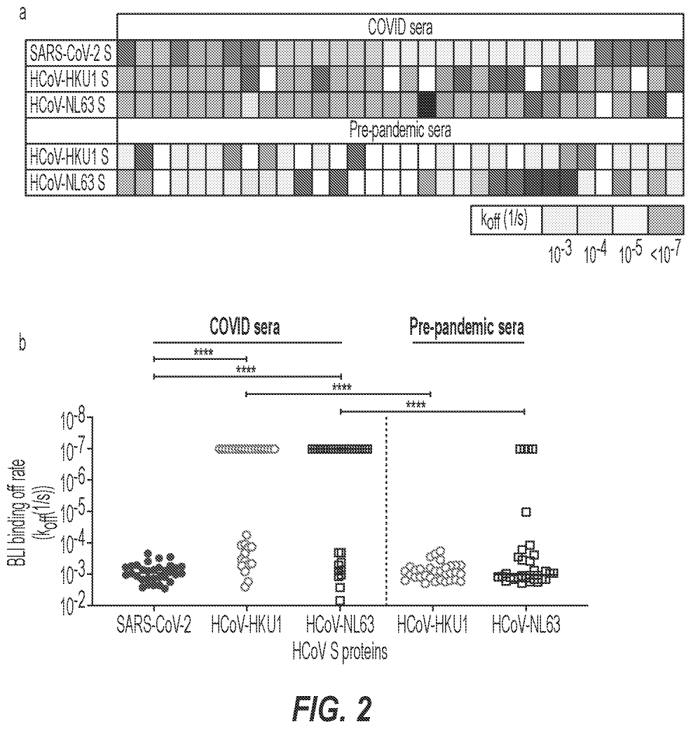

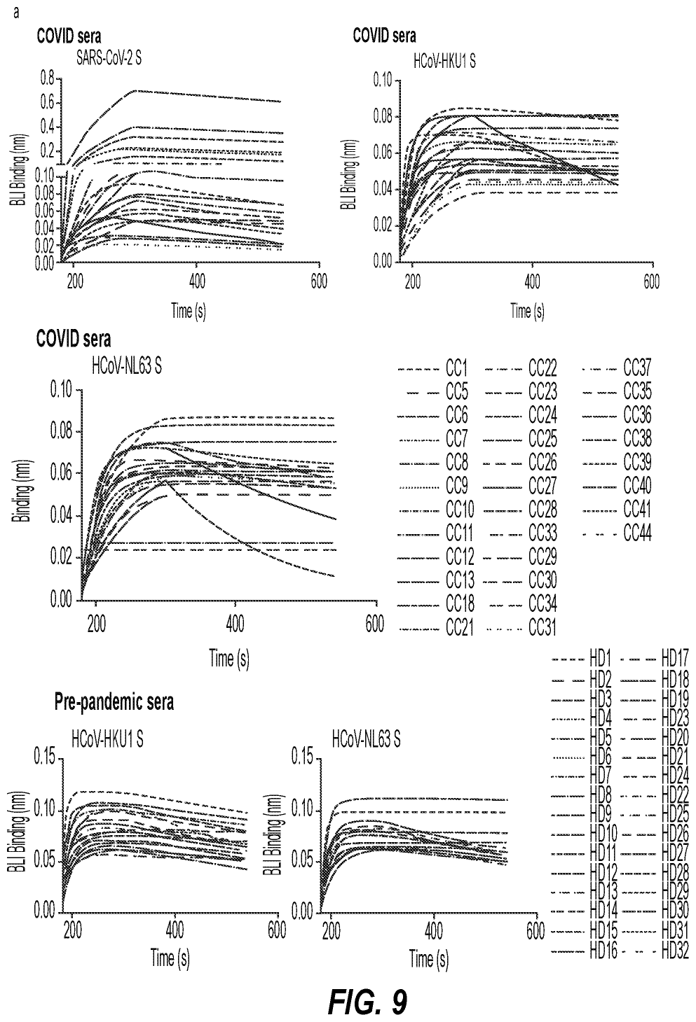

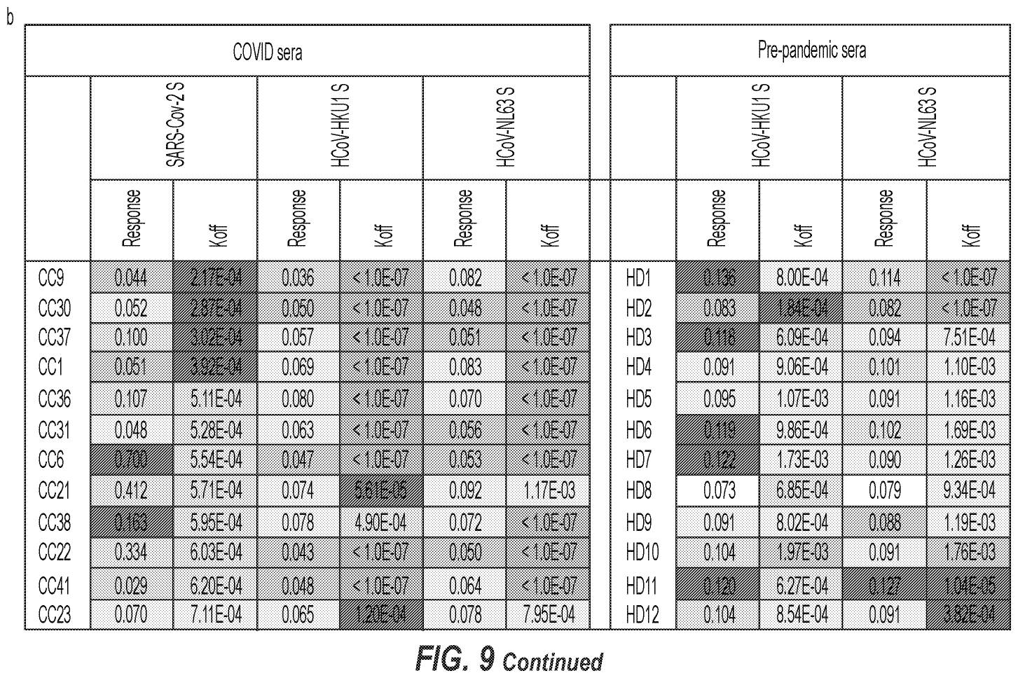

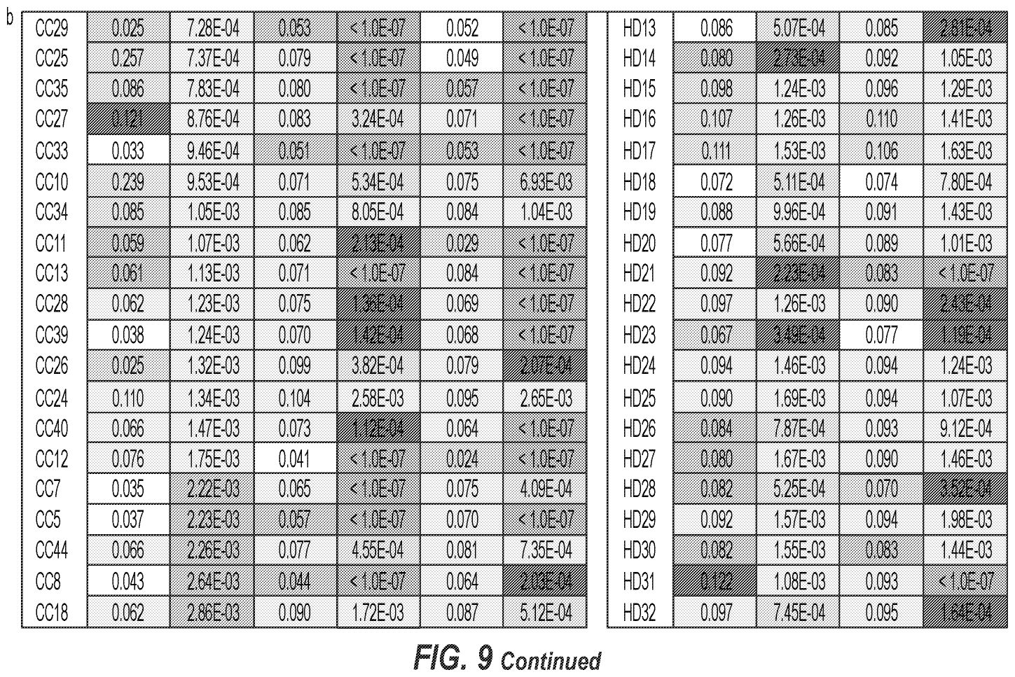

We were encouraged to look more closely at the Abs involved by Bio-Layer Interferometry (BLI). Polyclonal serum antibodies were used as analytes with biotinylated S proteins captured on streptavidin biosensors. Since the concentrations of the S protein specific polyclonal Abs in the sera are unknown, these measurements can provide an estimate of antibody dissociation off-rates (k_{off}, which is antibody concentration independent) but not binding constants. Slower dissociation off-rates would indicate greater affinity maturation of antibodies with a given S protein. It is important to note that the off-rates are likely associated with bivalent IgG binding (avidity) in the format used. Consistent with the notion of SARS-CoV-2 infection activating a recall of cross-reactive HCoV S specific Abs, the COVID sera Abs exhibited significantly slower off-rates with HCoV-HKU1 and HCoV-NL63 S-proteins compared to pre-pandemic sera Abs (FIG. 2A-B, FIG. 9).

### Example 2. Cross-Reactivity in Memory B Cells in COVID Individuals

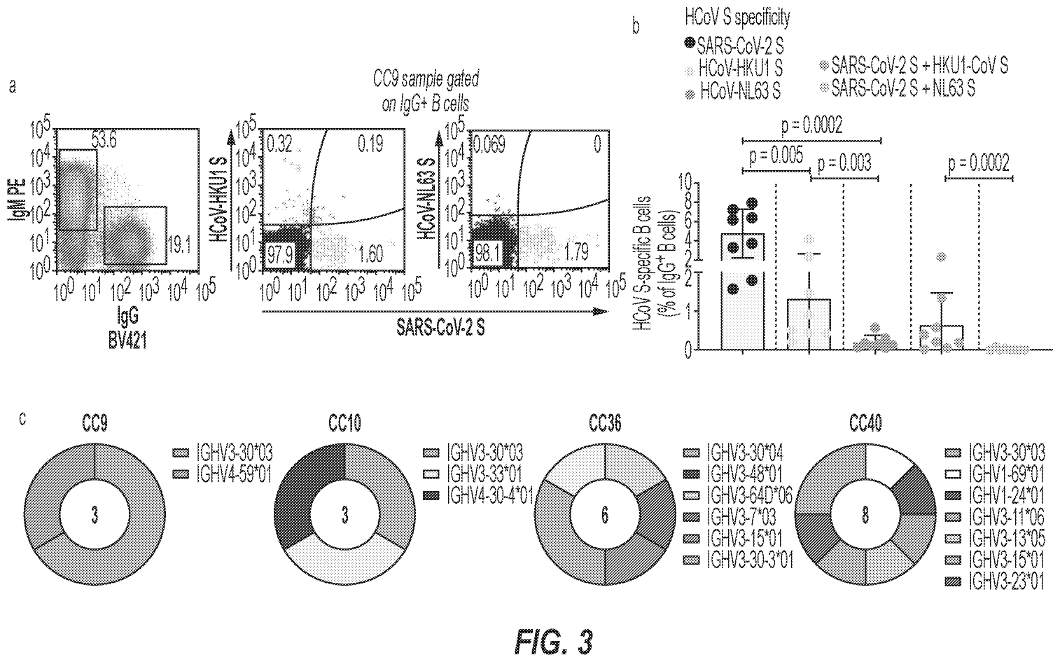

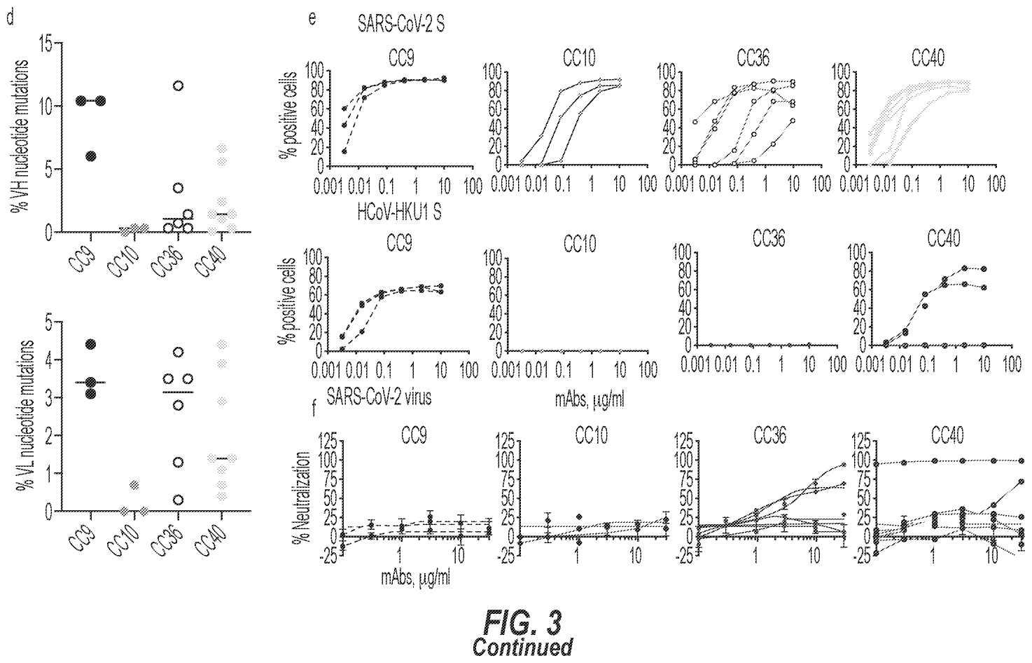

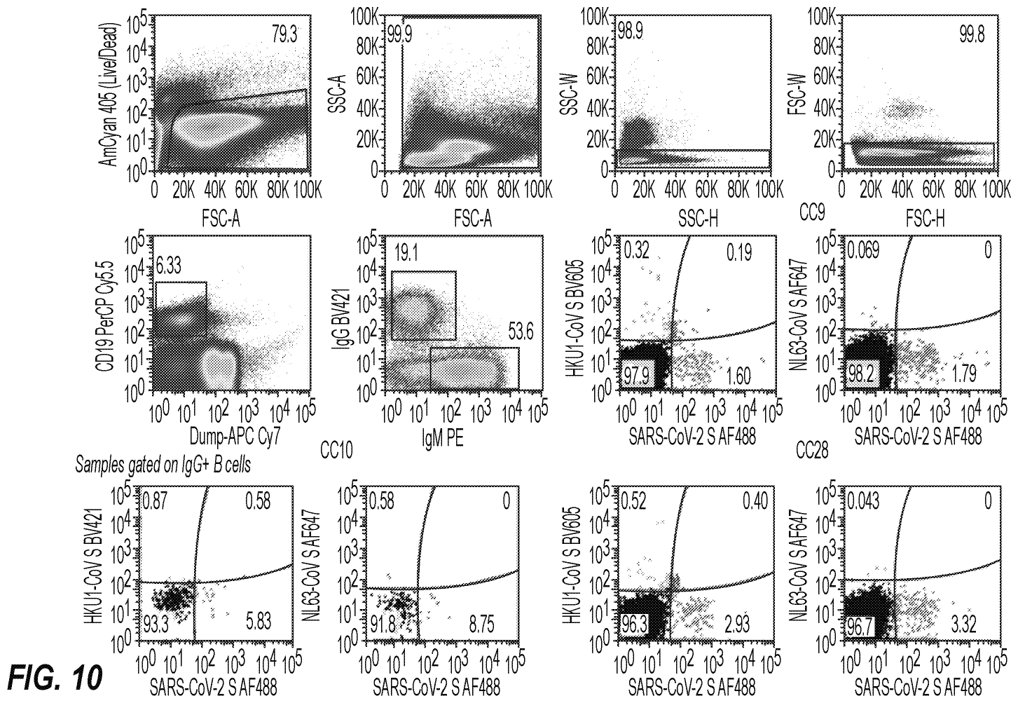

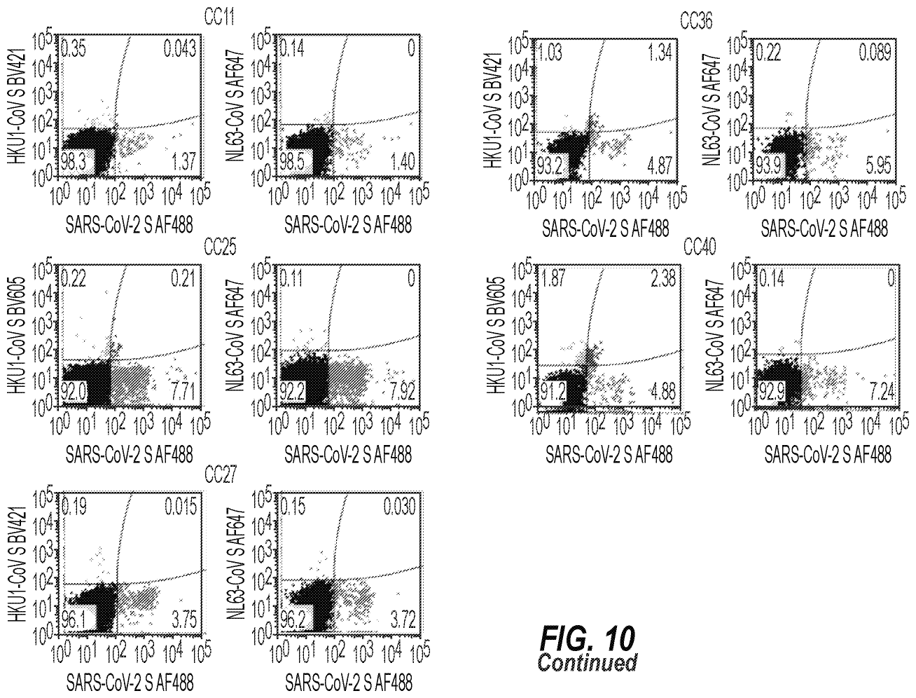

Having probed serum cross-reactivity between coronaviruses, we next investigated memory B cells in COVID individuals. We examined the reactivities of IgG+ memory B cells in 8 select COVID donors (based on differential binding to HCoV spikes (FIG. 1) with SARS-CoV-2, HCoV-HKU1 (b-HCoV) and HCoV-NL63 (α-HCoV) S-proteins by flow cytometry. Up to ˜8% SARS-CoV-2 S-protein, ˜4.3% HCoV-HKU1 S-protein and ˜0.6% for HCoV-NL63 S-protein-specific B cells were identified (FIG. 3A-B, FIG. 10) in a frequency pattern consistent with serum antibody binding titers.

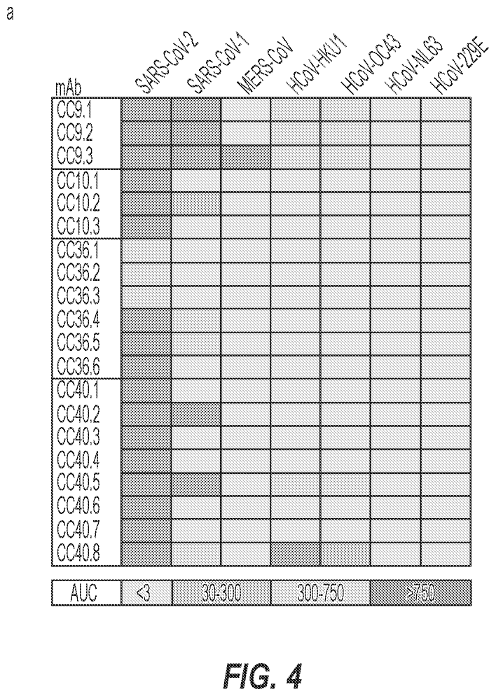

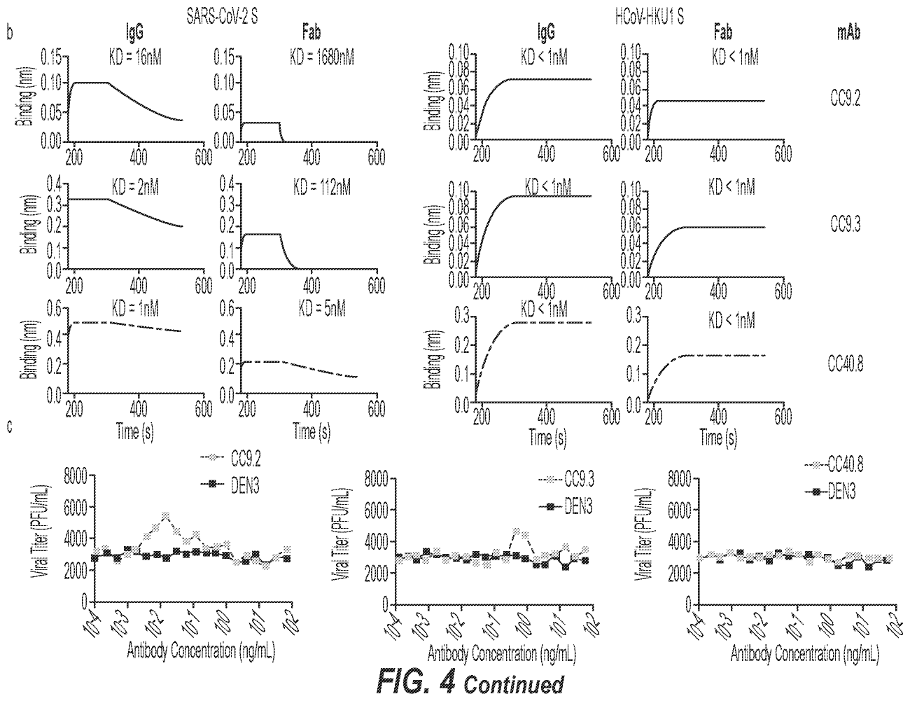

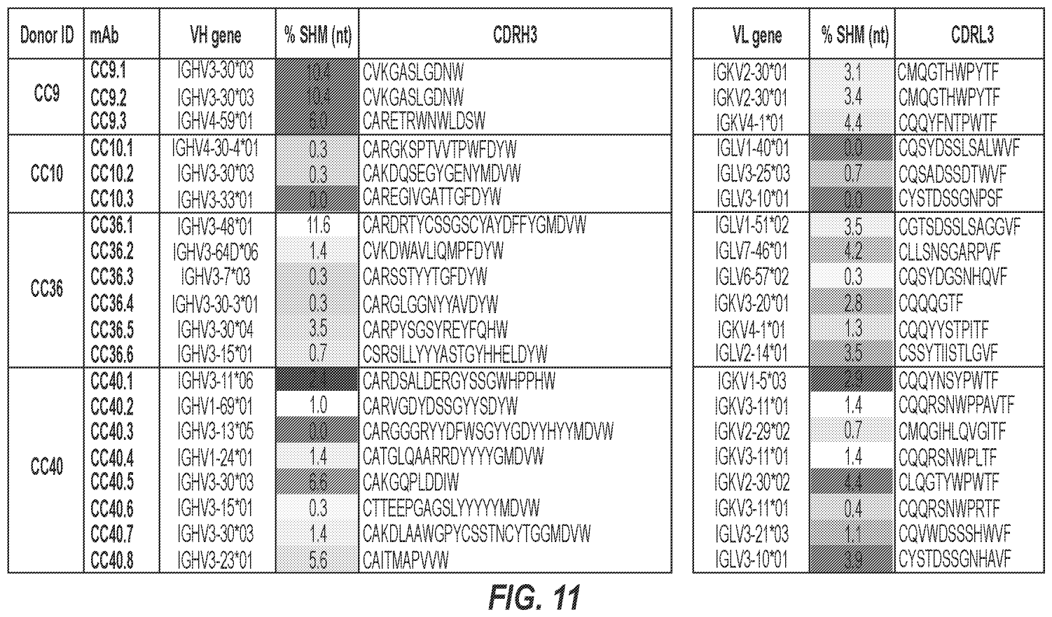

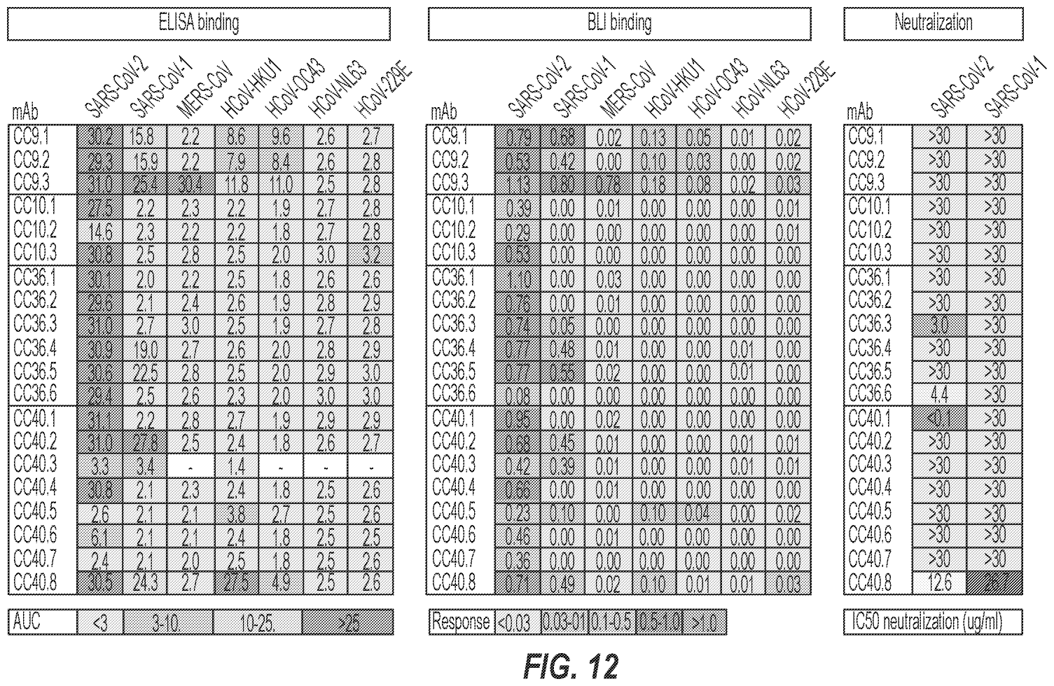

To probe the specificities of SARS-CoV-2/endemic HCoV cross-reactive Abs, we sorted single B cells for either SARS-CoV-2/HCoV-HKU-1 or SARS-CoV-2/HCoV-NL63 CoV S-protein double positivity. We isolated 20 S-protein-specific mAbs from 4 COVID donors, CC9 (n=3), CC10 (n=3), CC36 (n=6) and CC40 (n=8) (FIG. 3C, FIG. 11) but only 5 mAbs, 3 from the CC9 donor and 2 from the CC40 donor, exhibited cross-reactive binding with HCoV-HKU1 spike (FIG. 3E). Two of the cross-reactive mAbs from the CC9 donor (CC9.1 and CC9.2) were clonally related. All 5 of the SARS-CoV-2/HCoV-HKU-1 cross-reactive mAbs displayed binding to the genetically related b-HCoV, HCoV-OC43, spike but not to the α-HCoVs, HCoV-NL63 and HCoV-229E, spikes (FIG. 4A, FIG. 12). Notably, one mAb (CC9.3) exhibited binding to 5 out of the 7 HCoVs, including the MERS-CoV S-protein (FIG. 4A, FIG. 12) suggesting targeting of a highly conserved epitope on b-HCoV spikes. One of the 4 SARS-CoV-2/HKU1-CoV S cross-reactive mAbs (CC40.8) showed weak cross neutralization against SARS-CoV-2 and SARS-CoV-1 viruses (FIG. 12). Except for CC9.3 mAb, all cross-reactive mAbs were encoded by VH3 family gene heavy chains (FIGS. 11 and 12) and possessed 5.6-10.4% (median=6.6%) VH and 3.1-4.4% (median=3.9%) VL nucleotide SHMs (FIG. 3D, FIG. 11).

In principle, the SARS-CoV-2/HCOV-HKU1 S cross-reactive memory B cells could be pre-existing in the COVID donors and show cross-reactivity with SARS-CoV-2 or originate from the SARS-CoV-2 infection and show cross-reactivity with HCoV-HKU1 S protein. The levels of SHM in the 5 cross-reactive mAbs listed above argue for the first explanation. To gain further insight, we conducted BLI binding studies on the 3 cross-reactive mAbs, CC9.2, CC9.3 and CC40.8 (FIG. 4B). Both bivalent IgGs and monovalent Fabs showed enhanced binding affinity to HCoV-HKU1 S-protein compared to SARS-CoV-2 S-protein (FIG. 4B) again consistent with the notion that the Abs (BCRs) arise from a pre-existing HCoV-HKU1 S response. The serum and BCR data are then consistent. The data above suggests elevated serum levels of Abs to HCoV-HKU1 S-protein in COVID donors compared to pre-pandemic donors (FIG. 2A-B) is consistent with the notion that SARS-CoV-2 activates B cells expressing pre-existing HCoV-HKU1 S-protein specific BCRs to secrete the corresponding Abs.

### Example 3. Epitope Mapping

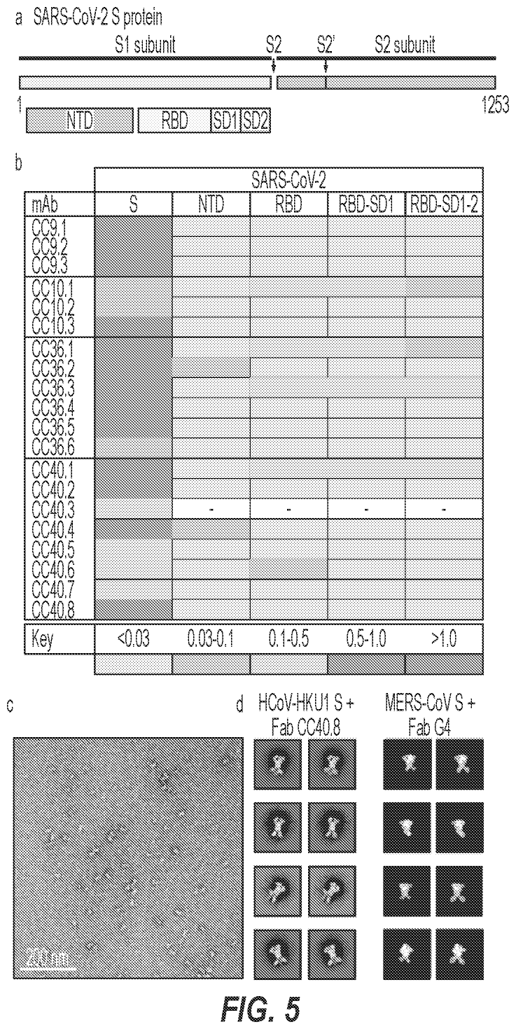

To map the epitope specificities of the cross-reactive mAbs, we evaluated binding to a number of fragments of the S-protein (FIG. 5A-B). Notably, all 5 of the SARS-CoV-2/HKU1-CoV cross-reactive mAbs failed to bind any of the S1 subunit domains or subdomains, suggesting targeting to the more conserved S2 subunit. To identify the cross-reactive neutralizing epitope recognized by mAb CC40.8, we conducted structural studies of the antibody with the HKU1-CoV S protein. Using single particle negative stain electron microscopy (nsEM) we observed that CC40.8 bound to the HCoV-HKU1 S trimer near the bottom of the S2 domain (FIG. 5C-D). The Fab density in the 2D class averages was blurry suggesting binding to a flexible surface exposed peptide. The flexibility also precluded further 3D reconstruction.

Despite the requirement of double positivity in the B cell sorting, 15/20 mAbs were largely specific for SARS-CoV-2. Again, like cross-reactive mAbs above, the vast majority of SARS-CoV-2 specific mAbs were encoded by VH3 family gene-encoded heavy chains (FIG. 3C, FIG. 11). Overall, the SARS-CoV-2 spike antigenic surface can be recognized by various human VH-gene families, but there is a bias toward the VH3-family gene-encoded antibodies and rational vaccine design strategies may take this feature into consideration. VH-germline gene specific bias for antigenic shapes is common and has been previously reported for many pathogen surfaces. Compared to the cross-reactive mAbs, the nucleotide SHM levels in SARS-CoV-2 specific mAbs were much lower (VH, 0-11.6% (median=0.7%) VL, 0-4.2% (median=1.3%)) (FIG. 3D, FIG. 11). 3 of the 15 SARS-CoV-2 S specific mAbs showed neutralization against SARS-CoV-2 virus, CC40.1 being the most potent (FIG. 3F, FIG. 12). Some of the SARS-CoV-2 specific mAbs exhibited cross-reactive binding with SARS-CoV-1 S protein but none neutralized SARS-CoV-1 virus.

### Example 4. Materials and Methods

Human cohort information: Plasma and PBMCs from convalescent COVID patients were kindly provided through the “Collection of Biospecimens from Persons Under Investigation for 2019-Novel Coronavirus Infection to Understand Viral Shedding and Immune Response Study” UCSD IRB #200236. Plasma from pre-pandemic donors were provided by Primary Infection Resource Consortium (PIRC) UCSD IRB #140093 and 191008. These donors were from an HIV-1 positive healthy cohort of individuals with well controlled HIV-1 and were on ARV. These pre-pandemic samples were collected from Apr. 18, 2019 to Mar. 3, 2020 before the spread of the pandemic in the US. Protocol was approved by the UCSD Human Research Protection Program. COVID patient samples were collected based on COVID-19 diagnosis regardless of gender, race, ethnicity, disease severity, or other medical conditions. The age and the ethnicity variables were relatively evenly distributed across the two human cohorts (COVID and pre-pandemic samples). The gender distribution in the pre-pandemic cohort could not be controlled due to the unavailability of the samples from female donors. The gender for individuals in the COVID cohort was evenly distributed. The summary of the demographic information of the COVID patients and pre-pandemic donors is listed in Table 2.

Plasmid construction for full-length and recombinant soluble proteins: To generate full-length human coronavirus plasmids, the spike genes were synthesized by GeneArt (Life Technologies). The SARS-CoV-1 (1255 amino acids; GenBank: AAP13567), SARS-CoV-2 (1273 amino acids; GenBank: MN908947), MERS-CoV (1353 amino acids; GenBank: APB87319.1), HCoV-HKU1 (1356 amino acids; GenBank: YP_173238.1), HCoV-OC43 (1361 amino acids; GenBank: AAX84792.1), HCoV-NL63 (1356 amino acids; GenBank: YP_003767.1) and HCoV-229E (1173 amino acids; GenBank: NP_073551.1) were cloned into the mammalian expression vector phCMV3 (Genlantis, USA) using PstI and BamH restriction sites. To express the soluble S ectodomain protein SARS-CoV-1 (residue 1-1190), SARS-CoV-2 (residue 1-1208), MERS-CoV (residue 1-1291), HCoV-HKU1 (residue 1-1295), HCoV-OC43 (residue 1-1300) and HCoV-NL63 (residue 1-1291), HCoV-229E (residue 1-1110), the corresponding DNA fragments were PCR amplified and constructed into vector phCMV3 using a Gibson assembly kit. To trimerize the soluble S proteins and stabilize them in the prefusion state, we incorporated a C-terminal T4 fibritin trimerization motif in the C-terminal of each constructs and two consecutive proline substitutions in the S2 subunit (Kirchdoerfer et al., Nature 531, 118-121, 2016; Pallesen et al., Proc. Natl. Acad. Sci. USA 114, E7348-E7357, 2017; and Wrapp et al., Science 367, 1260-1263, 2020). To be specific, the K968/V969 in SARS-CoV-1, the K986/V987 in SARS-CoV-2, the V1060/L1061 in MERS-CoV, the A1071/L1072 in HCoV-HKU1, the A1078/L1079 in HCoV-OC43, the S1052/I1053 in HCoV-NL63 and the T871/I872 in HCoV-229E were replaced by proline residues. Additionally, the S2 cleavage sites in each protein were replaced with a “GSAS” linker peptide. To facilitate the purification and biotin labeling of the soluble protein, the HRV-3C protease cleavage site, 6× HisTag, and AviTag spaced by GS-linkers were added to the C-terminus of the constructs, as needed. To express the SARS-CoV-2 N-terminal domain-NTD (residue 1-290), receptor-binding domain-RBD (residue 320-527), RBD-SD1 (residue 320-591), and RBD-SD1-2 (residue 320-681) subdomains, we amplified the DNA fragments by PCR reaction using the SARS-CoV-2 plasmid as template. All the DNA fragments were cloned into the vector phCMV3 (Genlantis, USA) in frame with the original secretion signal or the Tissue Plasminogen Activator (TPA) leader sequence. All the truncation proteins were fused to the C-terminal 6× HisTag, and AviTag spaced by GS-linkers to aid protein purification and biotinylation.

Expression and purification of the proteins: To express the soluble S ectodomain proteins of each human coronavirus and the truncated versions, the plasmids were transfected into FreeStyle293F cells (Thermo Fisher). For general production, 350 ug plasmids were transfected into 1 L FreeStyle293F cells at the density of 1 million cells/mL. We mixed 350 ug plasmids with 16 mL Transfectagro™ (Corning) and 1.8 mL 40K PEI (1 mg/mL) with 16 mL Transfectagro™ in separate 50 mL conical tubes. We filtered the plasmid mixture with 0.22 μm Steriflip™ Sterile Disposable Vacuum Filter Units (MilliporeSigma™) before combining it with the PEI mixture. After gently mixing the two components, the combined solution rested at room temperature for 30 min and was poured into 1 L FreeStyle293F cell culture. To harvest the soluble proteins, the cell cultures were centrifuged at 2500×g for 15 min on day 4 after transfection. The supernatants were filtered through the 0.22 μm membrane and stored in a glass bottle at 4° C. before purification. The His-tagged proteins were purified with the HisPur Ni-NTA Resin (Thermo Fisher). To eliminate nonspecific binding proteins, each column was washed with at least 3 bed volumes of wash buffer (25 mM Imidazole, pH 7.4). To elute the purified proteins from the column, we loaded 25 mL of the elution buffer (250 mM Imidazole, pH 7.4) at slow gravity speed (˜4 sec/drop). Proteins without His tags were purified with GNL columns (Vector Labs). The bound proteins were washed with PBS and then eluted with 50 mL of 1M Methyl α-D-mannopyranoside (Sigma M6882-500G) in PBS. By using Amicon tubes, we buffer exchanged the solution with PBS and concentrated the proteins. The proteins were further purified by size-exclusion chromatography using a Superdex 200 Increase 10/300 GL column (GE Healthcare). The selected fractions were pooled and concentrated again for further use.

Biotinylation of proteins: Random biotinylation of S proteins was conducted using EZ-Link NHS-PEG Solid-Phase Biotinylation Kit (Thermo Scientific #21440). 10 ul DMSO were added per tube for making concentrated biotin stock, 1 ul of which were diluted into 170 ul water before use. Coronavirus spike proteins were concentrated to 7-9 mg/ml using 100K Amicon tubes in PBS, then aliquoted into 30 ul in PCR tubes. 3 ul of the diluted biotin were added into each aliquot of concentrated protein and incubated on ice for 3 h. After reaction, buffer exchange for the protein was performed using PBS to remove excess biotin. BirA biotinylation of S proteins was conducted using BirA biotin-protein ligase bulk reaction kit (Avidity). Coronavirus S proteins with Avi-tags were concentrated to 7-9 mg/ml using 100K Amicon tubes in TBS, then aliquoted into 50 ul in PCR tubes. 7.5 ul of BioB Mix, 7.5 ul of Biotin200, and 5 ul of BirA ligase (3 mg/ml) were added per tube. The mixture was incubated on ice for 3 h, followed by size-exclusion chromatography to segregate the biotinylated protein and the excess biotin. The extend of biotinylation was evaluated by BioLayer Interferometry binding value using streptavidin biosensors.

CELISA binding: Binding of serum antibodies or mAbs to human coronavirus spike proteins expressed on HEK293T cell surface was determined by flow cytometry, as described previously (Walker et al., Science 326, 285-289, 2009). HEK293T cells were transfected with plasmids encoding full-length coronavirus spikes including SARS-CoV-1, SARS-CoV-2, MERS-CoV, HCoV-HKU1, HCoV-OC43, HCoV-NL63 and HCoV-229E. Transfected cells were incubated for 36-48 h at 37° C. Post incubation cells were trypsinized to prepare a single cell suspension and were distributed into 96-well plates. Serum samples were prepared as 3-fold serial titrations in FACS buffer (1× PBS, 2% FBS, 1 mM EDTA), starting at 1:30 dilution, 6 dilutions. 50 μl/well of the diluted samples were added into the cells and incubated on ice for 1 h. The plates were washed twice in FACS buffer and stained with 50 μl/well of 1:200 dilution of R-phycoerythrin (PE)-conjugated mouse anti-human IgG Fc antibody (SouthernBiotech #9040-09) and 1:1000 dilution of Zombie-NIR viability dye (BioLegend) on ice in dark for 45 min. After another two washes, stained cells were analyzed using flow cytometry (BD Lyrics cytometers), and the binding data were generated by calculating the percent (%) PE-positive cells for antigen binding using FlowJo 10 software. CR3022, a SARS-CoV-1 and SARS-CoV-2 spike binding antibody, and dengue antibody, DEN3, were used as positive and negative controls for the assay, respectively.

ELISA binding: 96-well half-area plates (Corning cat. #3690, Thermo Fisher Scientific) were coated overnight at 4° C. with 2 μg/ml of mouse anti-His-tag antibody (Invitrogen cat. #MA1-21315-1MG, Thermo Fisher Scientific) in PBS. Plates were washed 3 times with PBS plus 0.05% Tween20 (PBST) and blocked with 3% (wt/vol) bovine serum albumin (BSA) in PBS for 1 h. After removal of the blocking buffer, the plates were incubated with His-tagged spike proteins at a concentration of 5 μg/ml in 1% BSA plus PBS-T for 1.5 hr at room temperature. After a washing step, perturbed and lotus serum samples were added in 3-fold serial dilutions in 1% BSA/PBS-T starting from 1:30 and 1:40 dilution, respectively, and incubated for 1.5 hr. CR3022 and DEN3 human antibodies were used as a positive and negative control, respectively, and added in 3-fold serial dilutions in 1% BSA/PBS-T starting at 10 ug/ml. After the washes, a secondary antibody conjugated with alkaline phosphatase (AffiniPure goat anti-human IgG Fc fragment specific, Jackson ImmunoResearch Laboratories cat. #109-055-008) diluted 1:1000 in 1% BSA/PBS-T, was added to each well. After 1 h of incubation, the plates were washed and developed using alkaline phosphatase substrate pNPP tablets (Sigma cat. #S0942-200TAB) dissolved in a stain buffer. The absorbance was measured after 8, 20, and 30 minutes, and was recorded at an optical density of 405 nm (OD405) using a VersaMax microplate reader (Molecular Devices), where data were collected using SoftMax software version 5.4. The wells without the addition of serum served as a background control.

BioLayer Interferometry binding: An Octet K2 system (ForteBio) was used for performing the binding experiments of the coronavirus spike proteins with serum samples. All serum samples were prepared in Octet buffer (PBS plus 0.1% Tween20) as 1:40 dilution, random-biotinylated S proteins were prepared at a concentration of 100 nM. The hydrated streptavidin biosensors (ForteBio) first captured the biotinylated spike proteins for 60 s, then transferred into Octet buffer for 60 s to remove unbound protein and provide the baseline. Then, they were immersed in diluted serum samples for 120 s to provide association signal, followed by transferring into Octet buffer to test for disassociation signal for 240 s. The data generated was analyzed using the ForteBio Data Analysis software for correction and curve fitting, and for calculating the antibody dissociation rates (k_{off} values) or KD values for monoclonal antibodies.

Flow cytometry B cell profiling and mAb isolation with HCoV S proteins: Flow cytometry of PBMC samples from convalescent human donors were conducted following methods described previously (Yuan et, Science 369, 1119-1123, 2020; Walker et al., Science 326, 285-289; 2009; and Wu et al. Science 329, 856-861, 2010). Frozen human PBMCs were re-suspended in 10 ml RPMI 1640 medium (Thermo Fisher Scientific, #11875085) pre-warmed to 37° C. containing 50% fetal bovine serum (FBS). After centrifugation at 400×g for 5 minutes, the cells were resuspended in a 5 ml FACS buffer (PBS, 2% FBS, 2 mM EDTA) and counted. A mixture of fluorescently labeled antibodies to cell surface markers was prepared as 1:100 dilution that included antibodies specific for the T cell markers CD3(APC Cy7, BD Pharmingen #557757), CD4(APC-Cy7, Biolegend #317418) and CD8(APC-Cy7, BD Pharmingen #557760); B cell markers CD19 (PerCP-Cy5.5, Fisher Scientific #NC9963455), IgG(BV605, BD Pharmingen #563246) and IgM(PE); CD14(APC-Cy7, BD Pharmingen #561384, clone M5E2). The cells were incubated with the antibody mixture for 15 minutes on ice in the dark. The SARS-CoV-2 S protein was conjugated to streptavidin-AF488 (Life Technologies #S11223), the HCoV-HKU1 S protein to streptavidin-BV421 (BD Pharmingen #563259) and the HCoV-NL63 S protein to streptavidin-AF647 (Life Technologies #S21374). Following conjugation, each S protein-probe was added to the Ab-cell mixture and incubated for 30 minutes on ice in the dark. FVS510 Live/Dead stain (Thermo Fisher Scientific, #L34966) in the FACS buffer (1:300) was added to the cells and incubated on ice in the dark for 15 minutes. The stained cells were washed with FACS buffer and re-suspended in 500 μl of FACS buffer/10-20 million cells, passed through a 70 μm mesh cap FACS tube (Fisher Scientific, #08-771-23) and sorted using a Beckman Coulter Astrios sorter, where memory B cells specific to S protein proteins were isolated. In brief, after the gating of lymphocytes (SSC-A vs. FSC-A) and singlets (FSC-H vs. FSC-A), live cells were identified by the negative FVS510 Live/Dead staining phenotype, then antigen-specific memory B cells were distinguished with sequential gating and defined as CD3−, CD4−, CD8−, CD14−, CD19+, IgM− and IgG+. Subsequently, the S protein specific B cells were identified with the phenotype of AF488+BV421+ (SARS-CoV-2/HCoV-HKU1 S protein double positive) or AF488+AF647+ (SARS-CoV-2/HCoV-NL63 S protein double positive). Positive memory B cells were then sorted and collected at single cell density in 96-well plates. Downstream single cell IgG RT-PCR reactions were conducted using Superscript IV Reverse Transcriptase (Thermo Fisher, #18090050), random hexamers (Gene Link #26400003), Ig gene-specific primers, dNTP, Igepal, DTT and RNAseOUT (Thermo Fisher #10777019). cDNA products were then used in nested PCR for heavy/light chain variable region amplification with HotStarTaq Plus DNA Polymerase (QIAGEN #203643) and specific primer sets described previously (Tiller, J. Immunol. Methods 329, 112-124, 2008; and Doria-Rose et al., J. Virol. 90, 76-91, 2016). The second round PCR exploited primer sets for adding on the overlapping region with the expression vector, followed by cloning of the amplified variable regions into vectors containing constant regions of IgG1, Ig Kappa, or Ig Lambda using Gibson assembly enzyme mix (New England Biolabs #E2621L) after confirming paired amplified product on 96-well E gel (ThermoFisher #G720801). Gibson assembly products were finally transformed into competent E. coli cells and single colonies were picked for sequencing and analysis on IMGT V-Quest online tool (http://www.imgt.org) as well as downstream plasmid production for antibody expression.

Neutralization assay: Under BSL2/3 conditions, MLV-gag/pol and MLV-CMV plasmids were co-transfected into HEK293T cells along with full-length or variously truncated SARS-CoV1 and SARS-COV2 spike plasmids using Lipofectamine 2000 to produce single-round of infection competent pseudo-viruses. The medium was changed 16 hours post transfection. The supernatant containing MLV-pseudotyped viral particles was collected 48 h post transfection, aliquoted and frozen at −80° C. for neutralization assay. Pseudotyped viral neutralization assay was performed as previously described with minor modification (Modified from TZM-bl assay protocol; Sarzotti-Kelsoe et al., J. Immunol. Methods 409, 131-146, 2014). 293T cells were plated in advance overnight with DMEM medium +10% FBS+1% Pen/Strep+1% L-glutamine. Transfection was done with Opti-MEM transfection medium (Gibco, 31985) using Lipofectamine 2000. The medium was changed 12 hours after transfection. Supernatants containing the viruses were harvested 48 h after transfection. 1) Neutralization assay for plasma. plasma from COVID donors were heat-inactivated at 56° C. for 30 minutes. In sterile 96-well half-area plates, 25 μl of virus was immediately mixed with 25 μl of serially diluted (3×) plasma starting at 1:10 dilution and incubated for one hour at 37° C. to allow for antibody neutralization of the pseudotyped virus. 10,000 HeLa-hACE2 cells/well (in 50 ul of media containing 20 μg/ml Dextran) were directly added to the antibody virus mixture. Plates were incubated at 37° C. for 42 to 48 h. Following the infection, HeLa-hACE2 cells were lysed using 1× luciferase lysis buffer (25 mM Gly-Gly pH 7.8, 15 mM MgSO4, 4 mM EGTA, 1% Triton X-100). Luciferase intensity was then read on a Luminometer with luciferase substrate according to the manufacturer's instructions (Promega, PR-E2620). 2) Neutralization assay for monoclonal antibodies. In 96-well half-area plates, 25 ul of virus was added to 25 ul of five-fold serially diluted mAb (starting concentration of 50 ug/ml) and incubated for one hour before adding HeLa-ACE2 cell as mentioned above. Percentage of neutralization was calculated using the following equation: 100×(1−(MFI of sample−average MFI of background)/average of MFI of probe alone−average MFI of background)).

Antibody dependent enhancement assay: Ex vivo antibody dependent enhancement (ADE) quantification was measured using a focus reduction neutralization assay. Monoclonal antibodies were serially diluted in complete RPMI and incubated for 1 hour at 37° C. with SARS-CoV-2 strain USA-WA1/2020 (BEI Resources NR-52281) [MOI=0.01], in a BSL3 facility. Following the initial incubation, the mAb-virus complex was added in triplicate to 384-well plates seeded with 1E4 of K562 or Daudi cells and were incubated at 34° C. for 24 hours. 20 μL of the supernatant was transferred to a 384-well plate seeded with 2E3 HeLa-ACE2 cells and incubated for an additional 24 hours at 34° C. Plates were fixed with 25 ul of 8% formaldehyde for 1 hour at 34° C. Plates were washed 3 times with 1×PBS 0.05% Tween-20 following fixation. 10 μL of human polyclonal sera diluted 1:500 in Perm/Wash Buffer (BD Biosciences) was added to the plate and incubated at RT for 2 hours. The plates were then washed 3 times with 1×PBS 0.05% Tween-20 and stained with peroxidase goat anti-human Fab (Jackson Scientific, 109-035-006) diluted 1:2000 in Perm/wash buffer then incubated at RT for 2 hours. The plates were then washed 3 times with 1×PBS 0.05% Tween-20. 10 μL of Perm/Wash buffer was added to the plate then incubated for 15 minutes at RT. The Perm/Wash buffer was removed and 10 μL of TrueBlue peroxidase substrate (KPL) was added. The plates were incubated for 30 minutes at RT then washed once with milli-Q water. The FFU per well was then quantified using a compound microscope. The PFU/mL of the monocyte plate supernatant was calculated and graphed using Prism 8 software.

Negative Stain Electron Microscopy: The HCoV-HKU1 S protein was incubated with a 3-fold molar excess of Fab CC40.8 for 30 mins at room temperature and diluted to 0.03 mg/ml in 1× TBS pH 7.4. 3 μL of the diluted sample was deposited on a glow discharged copper mesh grid, blotted off, and stained for 55 seconds with 2% uranyl formate. Proper stain thickness and particle density was assessed on a FEI Morgagni (80 keV). The Leginon software was used to automate data collection on a FEI Tecnai Spirit (120 keV), paired a FEI Eagle 4k×4k camera. The following parameters were used: 52,000× magnification, −1.5 μm defocus, a pixel size of 2.06 Å, and a dose of 25 e⁻/Å². Micrographs were stored in the Appion database (Lander, J. Struct. Biol. 166, 95-102, 2009), particles were picked using DogPicker (Voss et al., J Struct Biol 166, 205-213, 2009), and a particle stack of 256 pixels was made. RELION 3.0 (Scheres, J. Struct. Biol. 180, 519-530, 2012) was used to generate the 2D class averages. The flexibility of the fab relative to the spike precluded 3D reconstruction.

Statistical Analysis: Statistical analysis was performed using Graph Pad Prism 8 for Mac, Graph Pad Software, San Diego, California, USA. Median area-under-the-curve (AUC) or reciprocal 50% binding (ID50) or neutralization (IC50) titers were compared using the non-parametric unpaired Mann-Whitney-U test. The correlation between two groups was determined by Spearman rank test. Data were considered statistically significant at *p<0.05, **p<0.01, ***p<0.001, and ****p<0.0001.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. An antibody or antigen-binding fragment thereof that specifically binds to a human coronavirus (HCoV) spike protein with the same binding specificity as that of a reference antibody, wherein the reference antibody comprises heavy chain CDR1, CDR2 and CDR3 sequences and light chain CDR1, CDR2 and CDR3 sequences respectively set forth as (1) SEQ ID NOs:10-13, EDK, and SEQ ID NO:14 (CC40.8), (2) SEQ ID NOs:15-18, KVS, and SEQ ID NO:19 (CC40.5), (3) SEQ ID NOs:20-23, KVS, and SEQ ID NO:24 (CC9.1/9.2), or (4) SEQ ID NOs:25-28, WAS, and SEQ ID NO:29 (CC9.3).

2. The antibody or antigen-binding fragment thereof of claim 1, comprising a heavy chain variable region comprising CDR1-3 sequences that are respectively identical to SEQ ID NOs:10-13; SEQ ID NOs:15-17; SEQ ID NOs:20-22; or SEQ ID NOs:25-27.

3. The antibody or antigen-binding fragment thereof of claim 2, further comprising light chain variable region comprising CDR1-3 sequences that are respectively identical to SEQ ID NO:13, EDK, and SEQ ID NO:14; SEQ ID NO:18, KVS, and SEQ ID NO: 19; SEQ ID NO:23, KVS, and SEQ ID NO:24; or (4) SEQ ID NO:28, WAS, and SEQ ID NO:29.

4. The antibody or antigen-binding fragment thereof of claim 1, comprising a light chain variable region comprising CDR1-3 sequences that are respectively identical to SEQ ID NO:13, EDK, and SEQ ID NO:14; SEQ ID NO: 18, KVS, and SEQ ID NO:19; SEQ ID NO:23, KVS, and SEQ ID NO:24; or (4) SEQ ID NO:28, WAS, and SEQ ID NO:29.

5. The antibody or antigen-binding fragment thereof of claim 1, comprising heavy chain CDR1, CDR2 and CDR3 sequences and light chain CDR1, CDR2 and CDR3 sequences that are respectively identical to (1) SEQ ID NOs:10-13, EDK, and SEQ ID NO:14 (CC40.8), or conservatively modified variant thereof; (2) SEQ ID NOs:15-18, KVS, and SEQ ID NO:19 (CC40.5), or conservatively modified variant thereof; (3) SEQ ID NOs:20-23, KVS, and SEQ ID NO:24 (CC9.1/9.2), or conservatively modified variant thereof; or (4) SEQ ID NOs:25-28, WAS, and SEQ ID NO:29 (CC9.3), or conservatively modified variant thereof.

6. The antibody or antigen-binding fragment thereof of claim 1, comprising heavy chain CDR1, CDR2 and CDR3 sequences and light chain CDR1, CDR2 and CDR3 sequences that are respectively identical to (1) SEQ ID NOs:10-13, EDK, and SEQ ID NO:14 (CC40.8); (2) SEQ ID NOs:15-18, KVS, and SEQ ID NO:19 (CC40.5); (3) SEQ ID NOs:20-23, KVS, and SEQ ID NO:24 (CC9.1/9.2); or (4) SEQ ID NOs:25-28, WAS, and SEQ ID NO:29 (CC9.3).

7. The antibody or antigen-binding fragment thereof of claim 6, comprising a heavy chain variable region and a light chain variable region that are substantially identical to or conservatively modified variants of (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively.

8. The antibody or antigen-binding fragment thereof of claim 6, comprising a heavy chain variable region and a light chain variable region that are at least 95% identical to (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively.

9. The antibody or antigen-binding fragment thereof of claim 6, comprising a heavy chain variable region and a light chain variable region that are at least 99% identical to (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively.

10. The antibody or antigen-binding fragment thereof of claim 6, comprising a heavy chain variable region and a light chain variable region that are respectively identical to (1) SEQ ID NOs:1 and 2; (2) SEQ ID NOs:3 and 4; (3) SEQ ID NOs:5 and 6; (4) SEQ ID NOs:5 and 7; or (5) SEQ ID NOs:8 and 9.

11. A polynucleotide encoding (1) an antibody heavy chain variable region comprising CDR1-3 sequences (HCDR1-3) that are respectively identical to SEQ ID NOs:10-13; SEQ ID NOs:15-17; SEQ ID NOs:20-22; or SEQ ID NOs:25-27; and/or (2) an antibody light chain variable region comprising CDR1-3 sequences (LCDR1-3) that are respectively identical to SEQ ID NO:14, EDK, and SEQ ID NO:14; SEQ ID NO:18, KVS, and SEQ ID NO:19; SEQ ID NO:23, KVS, and SEQ ID NO:24; or (4) SEQ ID NO:28, WAS, and SEQ ID NO:29.

12. The polynucleotide of claim 11, encoding an antibody heavy chain variable region and an antibody light chain variable region, wherein the HCDR1-3 and the LCDR1-3 are respectively identical to (1) SEQ ID NOs:10-13, EDK, and SEQ ID NO:14, (2) SEQ ID NOs:15-18, KVS, and SEQ ID NO:19, (3) SEQ ID NOs:20-23, KVS, and SEQ ID NO:24, or (4) SEQ ID NOs:25-28, WAS, and SEQ ID NO:29.

13. The polynucleotide of claim 12, wherein the HCDR1-3 and LCDR1-3 are respectively identical to SEQ ID NOs:10-13, EDK, and SEQ ID NO:14.

14. The polynucleotide of claim 12, wherein the heavy chain variable region and the light chain variable region are substantially identical to or conservatively modified variants of (1) SEQ ID NOs:1 and 2, respectively; (2) SEQ ID NOs:3 and 4, respectively; (3) SEQ ID NOs:5 and 6, respectively; (4) SEQ ID NOs:5 and 7, respectively; or (5) SEQ ID NOs:8 and 9, respectively.

15. The polynucleotide of claim 12, wherein the heavy chain variable region and the light chain variable region are respectively identical to (1) SEQ ID NOs:1 and 2; (2) SEQ ID NOs:3 and 4; (3) SEQ ID NOs:5 and 6; (4) SEQ ID NOs:5 and 7; or (5) SEQ ID NOs:8 and 9.

16. A pharmaceutical composition comprising a therapeutically effective amount of the antibody or antigen-binding fragment of claim 1 and a pharmaceutically acceptable carrier.

17. The pharmaceutical composition of claim 16, wherein the antibody or antigen-binding fragment comprises HCDR1-3 and LCDR1-3 that are respectively identical to SEQ ID NOs:10-13, EDK, and SEQ ID NO:14.

18. The pharmaceutical composition of claim 16, wherein the antibody or antigen-binding fragment comprises heavy chain variable region and the light chain variable region are respectively identical to SEQ ID NOs:1 and 2.

19. A kit comprising an antibody or antigen-binding fragment of claim 1.

20. A method of treating or ameliorating symptoms associated with coronavirus infections in a subject, comprising administering the pharmaceutical composition of claim 16 to a subject afflicted with infection by one or more human coronaviruses (HCoVs), thereby treating or ameliorating symptoms associated with coronavirus infections in the subject.

21. The method of claim 20, wherein the one or more HCoVs comprise SARS-CoV-2.

22. The method of claim 20, wherein the antibody or antigen-binding fragment comprises heavy chain CDR1, CDR2 and CDR3 sequences and light chain CDR1, CDR2 and CDR3 sequences that are respectively identical to SEQ ID NOs:10-13, EDK, and SEQ ID NO:14.

23. A method of diagnosing a coronavirus infection in a human subject, comprising (a) obtaining a biological sample from the subject, and (b) contacting the sample with an antibody or antigen-binding fragment of claim 1 to detect a specific binding between an antigen in the sample and the antibody or antigen-binding fragment, thereby diagnosing a coronavirus infection in the subject.

24. The method of claim 23, wherein the biological sample is a blood sample or a saliva sample.

25. The method of claim 23, wherein the antibody or antigen-binding fragment comprises heavy chain CDR1, CDR2 and CDR3 sequences and light chain CDR1, CDR2 and CDR3 sequences that are respectively identical to SEQ ID NOs:10-13, EDK, and SEQ ID NO:14.
